(19) United States
(12) Patent Application Publication
(10) Pub. No.: US 2016/0116478 A1
(43) Pub. Date: Apr. 28, 2016

(54) REAGENT INCLUDING ANTI-LGR6 ANTIBODIES FOR DETECTION AND DIAGNOSIS OF CANCER

(71) Applicant: ORDER-MADE MEDICAL RESEARCH INC., Chiba (JP)

(72) Inventors: Hiroyuki SATOFUKA, Chiba (JP); Yasufumi MURAKAMI, Chiba (JP)

(73) Assignee: ORDER-MADE MEDICAL RESEARCH INC., Kashiwa-shi, Chiba (JP)

(21) Appl. No.: 14/894,254

(22) PCT Filed: May 29, 2014
(86) PCT No.: PCT/JP2014/064902

§ 371 Date: Nov. 25, 2015

(30) Foreign Application Priority Data
May 30, 2013 (JP) 2013-114150

(51) Int. Cl. G01N 33/574; C07K 16/28; C07K 16/30

(52) U.S. Cl. CPC G01N 33/57492; C07K 16/30; C07K 16/28; G01N 2333/726; C07K 2317/76

(57) ABSTRACT

The object of the present invention is to provide a reagent for detection or diagnosis of cancer.

The present invention provides a reagent for detection or diagnosis of cancer, which comprises a monoclonal antibody against LGR6 or a fragment of the antibody.

### TECHNICAL FIELD

The present invention relates to a reagent for detection or diagnosis of cancer, which comprises an anti-LGR6 antibody.

### BACKGROUND ART

Cancer ranks high in the causes of death in the world. Above all, colorectal cancer is at a higher position in the mortality of cancer in Europe and North America. The number of colorectal cancer patients has been drastically increasing in recent years, and about 60,000 patients suffer from colorectal cancer every year in Japan. In the number of deaths classified by internal organ, colorectal cancer ranks third after gastric cancer and lung cancer. Likewise, breast cancer is a woman's cancer most commonly seen in the United Kingdom, the United States and Japan. The number of women suffering from breast cancer in Japan has now exceeded 40,000 and still tends to increase.

Colorectal cancer and breast cancer can be cured by surgery before reaching advanced stages. On the other hand, even in unresectable progressive or recurrent cases, treatment outcomes have been greatly improved by advances in drug therapy. However, the average survival period is about two years for progressive or recurrent colorectal cancer cases and is about one and a half years for breast cancer cases.

For cancer diagnosis, it is useful to conduct detection on the basis of body fluid components prior to tissue staining. In the case of colorectal cancer, the diagnostic method based on fecal occult blood reaction is now used widely. This technique is designed to check the presence of human hemoglobin to thereby diagnose the presence or absence of hemorrhage in the intestines and indirectly predict the onset of colorectal cancer.

The fecal occult blood reaction is a test easy to perform and is therefore widely used, although there is a problem in the usefulness of the test. For example, to make a positive diagnosis in the test using Hemoccult II (Astellas Pharma Inc., Japan), this test requires 20 mg hemorrhage per day in the colon and rectum. However, hemorrhage actually caused in colorectal cancer patients is considered to be 10 mg or less. For this reason, the sensitivity of the fecal occult blood reaction is around 26%, so that only approximately ¼ of actual colorectal cancer patients can be detected and therefore ¾ of the patients will be missed, as reported (Non-patent Document 1: Jama, Vol. 269, 1262-7, 1993). Further, only 8.3% of the subjects diagnosed as a positive in the test actually have colorectal cancer. Thus, the fecal occult blood reaction is a test method involving many false positive results and therefore still has problems.

In the case of breast cancer, breast X-ray examination (mammography) or ultrasonic diagnosis is conducted. It has been reported that the mortality of breast cancer is 15% to 20% lower in a group continuously receiving mammographic examination than in a group not receiving this examination. On the other hand, recent reports have shown that mammography has no effect in reducing the mortality in women in their forties where women suffering from breast cancer increase in number (Non-patent Document 2: Annals of Internal Medicine, Vol. 137, 305-312, 2002). This is because breast cancer cannot be detected due to high mammary gland density, and it has also been reported that young Japanese women are particularly unsuited to mammographic examination. For this reason, there is a demand for the development of a new diagnostic method.

In cancer diagnosis, when a subject is suspected to have cancer as a result of physical examination, urine analysis, biochemical analysis of blood, blood counting, chest X-ray examination and/or fecal occult blood testing, such a subject will undergo histological examination and immunohistochemical examination using a biopsy sample which is a portion taken from a lump-containing tissue, followed by morphological diagnosis on the stained images. In general, from the obtained tissue, thin sections are prepared and fixed, and then subjected to hematoxylin-eosin staining (HE staining).

In histological observation, disappearance of tissue-constituting basal cells and other events serve as important factors for diagnosing a subject as having cancer. However, basal cells may also be altered in benign lesions, and diagnosis based solely on HE staining involves many difficulties, so that basal cells in atypical lesions are diagnosed mainly by immunohistochemical staining. In immunohistochemical staining, cancer is evaluated by detection of epidermal growth factor receptor (EGFR) for colorectal cancer and epidermal growth factor receptor 2 (Her2/erbB2) for breast cancer (Patent Document 1: JP 2006-519376 A).

However, the frequency of occurrence evaluated by immunohistochemical staining varies among reports from 25% to 77% in colorectal cancer upon detection of EGFR (Non-patent Document 3: Modern Media, Vol. 55, No. 7, 2009 (in Japanese)). In breast cancer, the frequency of occurrence is 15% to 25% upon detection of Her2, which means that a limited percentage of patients can be diagnosed as being positive (Non-patent Document 4: Endocr Relat Cancer, Vol. 9, p. 75-85, 2002). Moreover, both EGFR and Her2 may be stained even in normal tissues, depending on the type of staining; and therefore a strictly normalized method is required to make a positive diagnosis.

Under these circumstances, there is a demand for the development of a new target molecule which is expressed at high rate in cancer patients and allows effect prediction for antibody drugs, and the development of a monoclonal antibody required to detect such a new target molecule.

Leucine-rich repeat containing G protein-coupled receptor 6 (LGR6) is a membrane protein composed of 967 amino acids and has a structure consisting of a seven-transmembrane region and an N-terminal long extracellular region.

LGR6 gene is disclosed as one of the genes whose expression is increased in gastric cancer patients when compared to normal tissues (Non-patent Document 5: Virchows Arch., Vol. 461, p. 355-365, 2012). Moreover, the results are also shown that when 481 cases of gastric cancer were analyzed by tissue arrays using LGR6-recognizing rabbit-derived anti-LGR6 polyclonal antibody (antibodies-online), 153 cases (32%) were found to be LGR6-positive (ibid).

On the other hand, among LGR family proteins, LGR4 and LGR5 are known to have homology with LGR6, although there are many differences between LGR6 and these molecules. Thus, they are recognized to be distinct proteins when used as target molecules for detection purposes, as discussed below.

First, the number of leucine-rich repeats (LRRs) is 15 in LGR4 and LGR6, whereas it is 16 in LGR5, so that they differ in their protein structure (FIG. 1).

In addition, LGR4, LGR5 and LGR6 bind to R-spondin 1, 2, 3 and 4 to thereby enhance Wnt/β-catenin signals (Non-patent Document 6: PLoS One, Vol. 7, e37137, 2012), but the respective molecules LGR4, LGR5 and LGR6 have different affinities for each of R-spondin 1, 2, 3 and 4. For example, LGR6 is reported to have the highest affinity for R-spondin 2, followed by R-spondin 3, 1 and 4 in this order. Namely, the respective LGR molecules are inferred to also differ in their effect due to differences in their affinity for R-spondin members (Non-patent Document 6: PLoS One, Vol. 7, e37137, 2012).

Moreover, as a result of analyzing the expression profiles of LGR4, LGR5 and LGR6 in gastric cancer, the positive rates for LGR4 and LGR6 are shown to be 43% and 32%, respectively (Non-patent Document 5: Virchows Arch., Vol. 461, p. 355-365, 2012). In contrast, LGR5 is shown to be positive in normal gastric stem cells and therefore cannot be used as a marker for gastric cancer. On the other hand, LGR5 is suggested to have a potential to be used as a marker for cancer stem cells in colorectal cancer (Non-patent Document 7: Science, Vol. 337, p. 730-735, 2012). Moreover, it is shown that LGR5 is also expressed in areas deemed to be stem cells in gastric cancer, although its strong expression is not seen in any other cancer areas. In contrast, LGR4 and LGR6 are shown to be expressed throughout cancer areas, including stem cells, in gastric cancer.

Further, LGR4, LGR5 and LGR6 are also reported to greatly differ in their intracellular mRNA levels, depending on the type of human cancer cell line (Non-patent Document 6: PLoS One, Vol. 7, e37137, 2012).

Namely, LGR6 differs from LGR4 and LGR5 in terms of expression patterns in cancers.

In addition, the homology (identity) in the full-length amino acid sequence is 55% between LGR6 and LGR4, and is 49% between LGR6 and LGR5. Likewise, the homology in ECD2 including a hinge region is 42% between LGR6 and LGR4, and is 51% between LGR6 and LGR5. Namely, the homology between LGR6 and LGR4 or LGR5 cannot be regarded as being high.

Thus, LGR6 is a protein completely distinct as a target molecule from LGR4 and LGR5.

JP 2010-532169 A (Patent Document 2) discloses a monoclonal antibody specifically binding to the extracellular domain of human LGR protein. This document also shows that the antibody disrupts the binding of R-spondin 1, 2, 3 and 4 proteins to LGR protein and/or disrupts the R-spondin-mediated activation of LGR signaling. Further, this document discloses LGR4, LGR5 and LGR6 as members of the LGR protein.

However, it is only LGR5 and antibodies against LGR5 for which analysis results are shown in the above document, and no analysis results are shown for LGR6, nor is there any description showing that an antibody against LGR6 was obtained. As described above, LGR6 is a protein completely distinct as a target molecule from LGR4 and LGR5; and hence the structure, properties, functions and others of an antibody against LGR6 are not obvious from the descriptions in the above document.

Moreover, evaluations conducted in the above document are limited only to some cancer cases, i.e., analysis using tumorigenic (TG) colon cancer cells isolated from human tumor and expression analysis on 3 cases of colon tumor. Namely, the above document does not mention that LGR6 is overexpressed in other cancers, e.g., in colorectal cancer and breast cancer.

As antibodies against LGR6, polyclonal antibodies are provided from several manufacturers. Commonly known examples include LS-A621 (LifeSpan BioSciences), ABIN122207 (GenWay Biotech) and NBP1-49696 (Novus Biological), each being prepared by using the N-terminal segment of LGR6 as an antigen. Other known examples include H00059352-A01 prepared by using amino acid residues 445-504 of LGR6 as an antigen (Abnova Corporation), an antibody prepared by using amino acid residues 456-548 as an antigen (Novus Biologicalsh), and L4169 prepared by using amino acid residues 516-528 as an antigen (Sigma-Aldrich).

As a monoclonal antibody binding to LGR6, EPR6874 (GenTax) is known as a rabbit monoclonal antibody. However, because of being prepared by using an intracellular domain as an antigen, this antibody is difficult to use for detection of living cancer cells. Moreover, in the case of immunostaining, fixing of cells and permeation treatment of their cell membrane will cause denaturation of the LGR6 protein, so that the detection sensitivity of the antibody will be reduced.

To use an antibody as a tool for target protein detection or disease diagnosis, a monoclonal antibody is desired for this purpose because it is important to ensure continuous and uniform production and provision. In general, a membrane protein such as LGR6, particularly a multi-transmembrane protein is difficult to solubilize, and it is difficult to derive a high titer antibody against such a membrane protein due to its high amino acid sequence homology among mammals. For these reasons, it is difficult to prepare an antibody which allows detection of a membrane protein with high sensitivity. Even when polyclonal antibodies are obtained from, e.g., the peripheral blood of immunized animals, the probability of successfully isolating antibody-producing cells is very low under the present circumstances.

### DISCLOSURE OF THE INVENTION

The present invention has been made under these circumstances, and the problem to be solved by the present invention is to provide an antibody which allows detection of cancer with high sensitivity, and a reagent for detection or diagnosis of cancer, which comprises such an antibody.

As a result of extensive and intensive efforts made to solve the above problem, the inventors of the present invention have succeeded in detecting cancer with higher sensitivity by using an antibody binding to LGR6, particularly the extracellular region of LGR6, when compared to conventional antibodies. This has led to the completion of the present invention.

Namely, the present invention is as follows.

(1) A reagent for detection or diagnosis of cancer, which comprises a monoclonal antibody against leucine-rich repeat containing G protein-coupled receptor 6 (LGR6) or a fragment of the antibody.
(2) The reagent according to (1) above, wherein the antibody or fragment thereof binds to the extracellular region of leucine-rich repeat containing G protein-coupled receptor 6 (LGR6).
(3) The reagent according to (2) above, wherein the extracellular region comprises the amino acid sequence shown in SEQ ID NO: 7, 8, 9 or 10.
(4) The reagent according to (1) above, wherein the cancer is at least one selected from the group consisting of colorectal cancer, breast cancer, uterine cancer, gastric cancer, thyroid cancer, pancreatic cancer, brain tumor, cervical cancer, esophageal cancer, tongue cancer, lung cancer, small intestinal cancer, duodenal cancer, bladder cancer, kidney cancer, liver cancer, prostate cancer, uterine cervical cancer, ovarian cancer, gallbladder cancer, pharyngeal cancer, sarcoma, melanoma, leukemia, lymphoma and multiple myeloma.
(5) The reagent according to (1) above, wherein the cancer is at least one selected from the group consisting of colorectal cancer, breast cancer, uterine cancer, gastric cancer, thyroid cancer, pancreatic cancer and leukemia.
(6) The reagent according to (1) above, wherein the cancer is colorectal cancer or breast cancer.
(7) A method for detection of cancer, which comprises the step of contacting a monoclonal antibody against leucine-rich repeat containing G protein-coupled receptor 6 (LGR6) or a fragment of the antibody with a biological sample taken from a subject to thereby detect LGR6 in the sample.
(8) A kit for detection of cancer, which comprises a monoclonal antibody against leucine-rich repeat containing G protein-coupled receptor 6 (LGR6) or a fragment of the antibody.
(9) A method for diagnosis of cancer, which comprises the step of contacting a monoclonal antibody against leucine-rich repeat containing G protein-coupled receptor 6 (LGR6) or a fragment of the antibody with a biological sample taken from a subject, and detecting LGR6 in the sample.
(10) A monoclonal antibody against leucine-rich repeat containing G protein-coupled receptor 6 (LGR6) or a fragment of the antibody for use in the detection or diagnosis of cancer.

The present invention allows detection of cancer with higher sensitivity when compared to conventional antibodies.

### BRIEF DESCRIPTION OF THE DRAWINGS

FIG. 1 shows a structural comparison of LGR4, LGR5 and LGR6, along with an alignment of their partial regions.

FIG. 2 shows the results obtained when antibodies reactive to the immunized antigen (ECD2) were evaluated by ELISA.

FIG. 3 shows the results obtained when the antibodies produced by the established hybridomas were evaluated by ELISA.

FIG. 4 shows the results obtained when the antibodies produced by the established hybridomas were evaluated by Western blotting.

FIG. 5 shows the results obtained when HT29 cells were subjected to immunocytological staining with the antibodies produced by the established hybridomas.

FIG. 6 shows the results obtained when culture supernatants containing ECD2-reactive antibodies were used and evaluated in a flow cytometer.

FIG. 7 shows the results obtained when Mu2C15 antibody was purified with Protein G and analyzed for its reactivity to HT29 cells in a flow cytometer.

FIG. 8A shows the results obtained when human colorectal cancer tissues were immunostained with the antibody produced by hybridoma cells under clone No. Mu2C15.

FIG. 8B is a graph showing the results of statistical analysis on the degree of staining in immunohistological staining when ranked as strongly positive (Strong), positive (Moderate), weakly positive (Weak) or negative (Negative).

FIG. 9 shows the results obtained when human breast cancer tissues were immunostained with the antibody produced by hybridoma cells under clone No. Mu2C15.

FIG. 10A shows the results obtained when breast cancer tissues at different stages were immunostained with clone No. Mu2C15 and HercepTest II.

FIG. 10B is a graph showing the results of statistical analysis on the degree of staining in immunohistological staining with HercepTest II. The results of determination are expressed as “3+,” “2+,” “1+” and “0” in the order from strong to weak signals.

FIG. 10C is a graph showing the results of statistical analysis on the degree of staining in immunohistological staining with clone No. Mu2C15. The results of determination are expressed as “3+,” “2+,” “1+” and “0” in the order from strong to weak signals.

FIG. 11 shows an affinity comparison between known monoclonal antibody (clone No. 2A3) binding to the extracellular domain of LGR6 and clone No. Mu2C15.

FIG. 12 shows the results analyzed for combinations of antibodies allowing detection of the antigen ECD2 by sandwich ELISA. “−” denotes an absorbance of 0.2 to 0.3, “+” denotes an absorbance of 0.3 to 0.45, “++” denotes an absorbance of 0.45 to 0.8, and “+++” denotes an absorbance of 0.8 or more.

FIG. 13 shows a standard curve obtained when ECD2 was detected using Mu2C15 as an immobilized antibody and Mu2C21 as a detection antibody.

FIG. 14A shows the nucleotide sequence and amino acid sequence of the heavy chain variable region (VH) in Mu2C15 antibody, along with the positions of the signal sequence, CDR1, CDR2 and CDR3.

FIG. 14B shows the nucleotide sequence and amino acid sequence of the light chain variable region (VL) in Mu2C15 antibody, along with the positions of the signal sequence, CDR1, CDR2 and CDR3.

FIG. 15 shows the results compared for the reactivity of Mu2C15 antibody to partial peptides of LGR4, LGR5 and LGR6.

### BEST MODES FOR CARRYING OUT THE INVENTION

The present invention will be described in more detail below. The following embodiments are illustrated to describe the present invention, and it is not intended to limit the present invention only to these embodiments. The present invention can be implemented in various modes, without departing from the spirit of the present invention. Moreover, this specification incorporates the contents disclosed in the specification and drawings of Japanese Patent Application No. 2013-114150 (filed on May 30, 2013), based on which the present application claims priority.

### 1. Summary

The present invention relates to a reagent for detection or diagnosis of cancer, which comprises a monoclonal antibody against leucine-rich repeat containing G protein-coupled receptor 6 (LGR6) or a fragment of the antibody.

As a result of extensive and intensive efforts made with a focus on LGR6 which is a transmembrane protein, the inventors of the present invention have found that LGR6 is overexpressed in cancer, particularly in colorectal cancer and breast cancer. Moreover, the inventors of the present invention have succeeded in preparing a monoclonal antibody against LGR6, which allows detection of cancer with extremely high sensitivity and high accuracy when compared to conventionally known antibodies. The antibody of the present invention allows detection of cancer with high sensitivity even at an early stage of cancer progression and also allows detection of living cancer cells because of being capable of binding to the extracellular region of LGR6, which in turn allows detection of cancer cells present in blood samples, by way of example. Namely, when using the antibody of the present invention, cancers which have been undetectable by conventional methods can be detected in a simple manner with high sensitivity and high accuracy, so that the antibody of the present invention is very useful for detection or diagnosis of cancer. The present invention has been completed on the basis of such findings.

### 2. Antibody Against LGR6 (Anti-LGR6 Antibody)

### (1) Antigen Preparation

LGR6 intended in the present invention may be derived from any mammal, and examples of such a mammal include mice, rats, rabbits, goats, monkeys and humans, with mice, rats and humans being preferred, and with humans being more preferred.

In the present invention, the amino acid sequences of mouse, rat and human LGR6 are shown in SEQ ID NOs: 2, 4 and 6, respectively. Likewise, the nucleotide sequences of DNAs encoding mouse, rat and human LGR6 are shown in SEQ ID NOs: 1, 3 and 5, respectively. These amino acid sequences and nucleotide sequences have been registered in the GenBank database under the accession numbers (Accession Nos.) given to the respective sequences.

Amino acid sequence of mouse LGR6: NP_001028581.1 (SEQ ID NO: 2)

Amino acid sequence of rat LGR6: XP_001062538.1 (SEQ ID NO: 4)

Amino acid sequence of human LGR6: NP_001017403.1 (SEQ ID NO: 6)

Nucleotide sequence of DNA encoding mouse LGR6: NM_001033409.3 (SEQ ID NO: 1)

Nucleotide sequence of DNA encoding rat LGR6: XM_001062538.3 (SEQ ID NO: 3)

Nucleotide sequence of DNA encoding human LGR6: NM_001017403.1 (SEQ ID NO: 5)

As an antigen, it is possible to use a polypeptide or peptide (also collectively referred to as a peptide) comprising at least a part (whole or a part) of the amino acid sequence of LGR6, preferably a peptide comprising at least a part (whole or a part) of the amino acid sequence of the extracellular region of LGR6.

The extracellular region of LGR6 refers to a region covering leucine-rich repeats (LRRs) and a hinge region connecting LRRs to transmembrane domains. Leucine-rich repeats refer to a structure in which a region composed of about 25 amino acid residues rich in leucine is located repeatedly. In the case of LGR6, the number of repeats is 15.

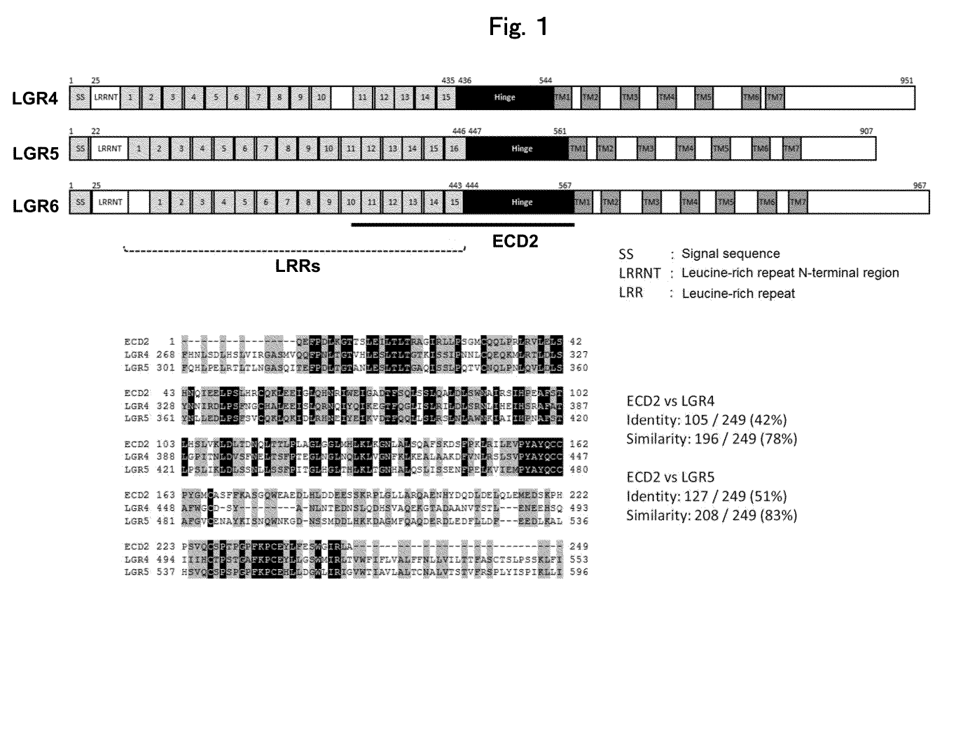

For example, in the case of human LGR6, the extracellular region of LGR6 corresponds to a region (SEQ ID NO: 7) consisting of amino acids at positions 25 to 567 in the amino acid sequence shown in SEQ ID NO: 6, LRRs correspond to a region (SEQ ID NO: 8) consisting of amino acids at positions 91 to 443 in the amino acid sequence shown in SEQ ID NO: 6, and the hinge region corresponds to a region (SEQ ID NO: 10) consisting of amino acids at positions 444 to 567 in the amino acid sequence shown in SEQ ID NO: 6 (FIG. 1). In the case of LGR6 derived from other animals, regions corresponding to these regions are intended.

The peptide to be used as an antigen is not limited in any way as long as it is at least a part of LGR6. Preferred is at least a part of the extracellular region of LGR6, e.g., at least a part of a region consisting of amino acids at positions 25 to 567 (SEQ ID NO: 7), a region consisting of amino acids at positions 91 to 567 (SEQ ID NO: 8), a region consisting of amino acids at positions 319 to 567 (SEQ ID NO: 9) or a region consisting of amino acids at positions 444 to 567 (SEQ ID NO: 10) in the amino acid sequence of LGR6 shown in SEQ ID NO: 6. Among them, more preferred is at least a part of the region consisting of amino acids at positions 319 to 567 or the region consisting of amino acids at positions 444 to 567, and particularly preferred is at least a part of the region consisting of amino acids at positions 319 to 567. In the present invention, a protein consisting of amino acids at positions 319 to 567 in the amino acid sequence of LGR6 shown in SEQ ID NO: 6 is also referred to as “ECD2.”

Moreover, LGR6 has mutants which are derived from the same mRNA but differ in their translation initiation site. For example, human LGR6 has mutants such as a peptide (SEQ ID NO: 11) lacking N-terminal 43 amino acids and having different amino acids at positions 44 to 71 from those of the wild-type (SEQ ID NO: 6), a peptide (SEQ ID NO: 12) lacking N-terminal 52 amino acids and having different amino acids at positions 53 to 70 from those of the wild-type (SEQ ID NO: 6), etc.

LGR6 intended in the present invention also includes these mutants.

LGR6 may be naturally occurring LGR6 purified from mouse, rat, human or other animal tissues or cells, or alternatively, may be genetically engineered LGR6. For example, a biological sample known to contain LGR6 may be fractionated into a soluble fraction and an insoluble fraction by using any type of surfactant such as Triton-X, Sarkosyl, etc. The insoluble fraction may further be dissolved in, e.g., urea or guanidine hydrochloride and then bound to any type of column such as a heparin column or crosslinked resin to thereby obtain LGR6.

Alternatively, the peptide to be used as an antigen may be prepared by chemical synthesis or by synthesis through genetic engineering procedures using E. coli or the like. Techniques well known to those skilled in the art may be used for this purpose.

For chemical synthesis, the peptide may be synthesized by well-known peptide synthesis techniques. Moreover, the synthesis may be accomplished by applying either solid phase synthesis or liquid phase synthesis. A commercially available peptide synthesizer (e.g., PSSM-8, Shimadzu Corporation, Japan) may also be used for this purpose.

For peptide synthesis through genetic engineering procedures, DNA encoding the peptide is first designed and synthesized. The design and synthesis may be accomplished, for example, by PCR techniques using a vector or the like containing the full-length LGR6 gene as a template and using primers which have been designed to allow synthesis of a desired DNA region. Then, the above DNA may be ligated to an appropriate vector to obtain a recombinant vector for protein expression, and this recombinant vector may be introduced into a host, such that a desired gene can be expressed therein, thereby obtaining a transformant (Sambrook J. et al., Molecular Cloning, A Laboratory Manual, 3rd edition, Cold Spring Harbor Laboratory Press, 2001).

As a vector, a phage or plasmid which is autonomously replicable in host microorganisms is used. Further, it is also possible to use an animal virus or insect virus vector. To prepare a recombinant vector, purified DNA may be cleaved with an appropriate restriction enzyme and ligated to a vector by being inserted into, e.g., an appropriate restriction enzyme site in the vector DNA. There is no particular limitation on the host for use in transformation as long as it is capable of expressing a desired gene. Examples include bacteria (e.g., E. coli, Bacillus subtilis), yeast, animal cells (e.g., COS cells, CHO cells), insect cells or insects. It is also possible to use a mammal (e.g., goat) as a host. Procedures for introduction of a recombinant vector into a host are known.

Moreover, the above transformant may be cultured, and a peptide for use as an antigen may be collected from the cultured product. The term “cultured product” is intended to mean either (a) a culture supernatant or (b) cultured cells or cultured microorganisms or a homogenate thereof.

After culture, when the desired peptide is produced within microorganisms or cells, the microorganisms or cells may be homogenized to thereby extract the peptide. Alternatively, when the desired peptide is produced outside microorganisms or cells, the cultured solution may be used directly or treated by centrifugation or other techniques to remove the microorganisms or cells. Then, the desired peptide may be isolated and purified by biochemical techniques commonly used for isolation and purification of peptides, as exemplified by ammonium sulfate precipitation, gel filtration, ion exchange chromatography, affinity chromatography and so on, which may be used either alone or in combination as appropriate.

In the present invention, the peptide to be used as an antigen may also be obtained by in vitro translation using a cell-free synthesis system. In this case, it is possible to use two methods, i.e., a method in which RNA is used as a template and a method in which DNA is used as a template (transcription/translation). As a cell-free synthesis system, a commercially available system may be used, as exemplified by an Expressway™ system (Invitrogen), etc.

The peptide obtained as described above may also be linked to an appropriate carrier protein such as bovine serum albumin (BSA), keyhole limpet hemocyanin (KLH), human thyroglobulin, avian gamma globulin, etc.

Moreover, the antigen intended in the present invention encompasses not only (a) a polypeptide comprising the amino acid sequence shown in SEQ ID NO: 2, 4, 6, 7, 8, 9, 10, 11 or 12, but also (b) a polypeptide comprising an amino acid sequence with deletion, substitution or addition of one or several amino acids in the amino acid sequence shown in SEQ ID NO: 2, 4, 6, 7, 8, 9, 10, 11 or 12 and (c) a polypeptide comprising an amino acid sequence sharing a homology of 70% or more with the amino acid sequence shown in SEQ ID NO: 2, 4, 6, 7, 8, 9, 10, 11 or 12.

In the context of the present invention, the “polypeptide comprising the amino acid sequence shown in SEQ ID NO: 2, 4, 6, 7, 8, 9, 10, 11 or 12” includes a polypeptide consisting of the amino acid sequence shown in SEQ ID NO: 2, 4, 6, 7, 8, 9, 10, 11 or 12.

Moreover, the “amino acid sequence with deletion, substitution or addition of one or several amino acids in the amino acid sequence shown in SEQ ID NO: 2, 4, 6, 7, 8, 9, 10, 11 or 12” includes, for example:

(i) an amino acid sequence with deletion of 1 to 10 amino acids (e.g., 1 to 5 amino acids, preferably 1 to 3 amino acids, more preferably 1 or 2 amino acids, even more preferably a single amino acid) in the amino acid sequence shown in SEQ ID NO: 2, 4, 6, 7, 8, 9, 10, 11 or 12;

(ii) an amino acid sequence with substitution of other amino acids for 1 to 10 amino acids (e.g., 1 to 5 amino acids, preferably 1 to 3 amino acids, more preferably 1 or 2 amino acids, even more preferably a single amino acid) in the amino acid sequence shown in SEQ ID NO: 2, 4, 6, 7, 8, 9, 10, 11 or 12;

(iii) an amino acid sequence with addition of 1 to 10 amino acids (e.g., 1 to 5 amino acids, preferably 1 to 3 amino acids, more preferably 1 or 2 amino acids, even more preferably a single amino acid) to the amino acid sequence shown in SEQ ID NO: 2, 4, 6, 7, 8, 9, 10, 11 or 12; and

(iv) an amino acid sequence mutated by any combination of (i) to (iii) above.

In addition, LGR6 intended in the present invention encompasses not only a polypeptide comprising the amino acid sequence shown in SEQ ID NO: 2, 4, 6, 7, 8, 9, 10, 11 or 12, but also a polypeptide comprising an amino acid sequence sharing a homology (identity) of 70% or more with the amino acid sequence shown in SEQ ID NO: 2, 4, 6, 7, 8, 9, 10, 11 or 12. Such a polypeptide also includes those comprising amino acid sequences sharing a homology of about 70% or more, 75% or more, about 80% or more, 85% or more, 90% or more, 95% or more, 96% or more, 97% or more, 98% or more, or 99% or more with the amino acid sequence shown in SEQ ID NO: 2, 4, 6, 7, 8, 9, 10, 11 or 12 (i.e., amino acid sequences substantially equivalent to the amino acid sequence shown in SEQ ID NO: 2, 4, 6, 7, 8, 9, 10, 11 or 12). For homology determination, it is possible to use homology search tools such as FASTA, BLAST, PSI-BLAST and so on in a homology search site on the Internet (e.g., DNA Data Bank of Japan (DDBJ)). Alternatively, it is also possible to conduct a BLAST search in the National Center for Biotechnology Information (NCBI).

To prepare the above mutation-carrying proteins, mutations may be introduced into a gene (DNA) encoding the protein by using a kit for mutation introduction based on site-directed mutagenesis (e.g., Kunkel method or Gapped duplex method), as exemplified by a QuikChange™ Site-Directed Mutagenesis Kit (Stratagene), GeneTailor™ Site-Directed Mutagenesis Systems (Invitrogen), TaKaRa Site-Directed Mutagenesis Systems (e.g., Mutan-K, Mutan-Super Express Km; Takara Bio Inc., Japan). Alternatively, it is also possible to use techniques for site-directed mutagenesis as described in “Molecular Cloning, A Laboratory Manual (4th edition)” (Cold Spring Harbor Laboratory Press (2012)), etc.

In the present invention, the gene to be introduced into cells or the like may be a gene encoding LGR6 or a partial fragment thereof or a mutated peptide thereof. As such a gene, it is possible to use a gene comprising the nucleotide sequence shown in SEQ ID NO: 1, 3 or 5, by way of example.

Alternatively, the gene to be introduced into cells or the like may also be a gene comprising a nucleotide sequence or a partial sequence thereof, which is hybridizable under stringent conditions with a sequence complementary to the nucleotide sequence shown in SEQ ID NO: 1, 3 or 5.

In the context of the present invention, the term “stringent conditions” may be any of low stringent conditions, moderately stringent conditions and high stringent conditions. “Low stringent conditions” refer to, for example, conditions of 5×SSC, 5×Denhardt's solution, 0.5% SDS, 50% formamide and 32° C. Likewise, “moderately stringent conditions” refer to, for example, conditions of 5×SSC, 5×Denhardt's solution, 0.5% SDS, 50% formamide and 42° C. “High stringent conditions” refer to, for example, conditions of 5×SSC, 5×Denhardt's solution, 0.5% SDS, 50% formamide and 50° C. Under these conditions, it can be expected that DNA having a higher homology is more efficiently obtained at a higher temperature. However, the stringency of hybridization would be affected by a plurality of factors, including temperature, probe concentration, probe length, ionic strength, reaction time, salt concentration and so on. Those skilled in the art would be able to achieve the same stringency by selecting these factors as appropriate.

Moreover, the gene encoding LGR6 for use in the present invention also encompasses a gene comprising a nucleotide sequence sharing a homology of 60% or more with the nucleotide sequence shown in SEQ ID NO: 1, 3 or 5. Such a gene may be exemplified by a gene sharing a homology of 60% or more, 70% or more, 80% or more, 85% or more, 90% or more, 95% or more, 96% or more, 97% or more, 98% or more, or 99% or more with the nucleotide sequence shown in SEQ ID NO: 1, 3 or 5, as calculated by homology search software such as FASTA or BLAST using default parameters.

The above gene encoding LGR6 or a mutant thereof can be obtained, for example, on the basis of the nucleotide sequences found in the NCBI GenBank database by using known genetic engineering procedures as described in “Molecular Cloning, A Laboratory Manual (4th edition)” (Cold Spring Harbor Laboratory Press (2012)), etc.

### (2) Preparation of Polyclonal Antibodies

The thus prepared LGR6 or partial peptide thereof is administered directly or together with a carrier or diluent to immunize non-human mammals (e.g., rabbits, dogs, guinea pigs, mice, rats, goats). The amount of an antigen to be administered per animal is 1 μg to 10 mg in the case of using an adjuvant. Examples of an adjuvant include Freund's complete adjuvant (FCA), Freund's incomplete adjuvant (FIA), aluminum hydroxide adjuvant and so on. Immunization is accomplished primarily by injection via the intravenous, subcutaneous or intraperitoneal route, etc. Moreover, the interval between immunizations is not limited in any way, and immunization may be repeated twice to 20 times, preferably 5 to 15 times, at intervals of several days to several weeks, preferably at intervals of 1 or 2 weeks. Those skilled in the art would be able to determine the interval between immunizations in consideration of the resulting antibody titers. Preferably, at the time of repeating subcutaneous immunization 3 to 4 times, blood is sampled and measured for antibody titers. Antibody titers in serum may be measured by ELISA (enzyme-linked immunosorbent assay), EIA (enzyme immunoassay), radioimmunoassay (RIA), etc. After antibody titers have been confirmed to rise sufficiently, blood may be collected completely and treated in a commonly used manner to separate and purify antibodies. For separation and purification, known techniques such as salting out with ammonium sulfate, ion exchange chromatography, gel filtration chromatography, affinity chromatography and so on may be selected as appropriate or used in combination. More specifically, serum containing desired antibodies may be passed through a column on which proteins other than LGR6 have been immobilized, and fractions passing through the column may be collected to thereby obtain polyclonal antibodies with improved specificity to LGR6.

### (3) Preparation of Monoclonal Antibodies

### (i) Collection of Antibody-Producing Cells

As in the case of polyclonal antibody preparation, LGR6 or a partial peptide thereof (e.g., ECD2) is administered directly or together with a carrier or diluent to immunize non-human mammals. The amount of an antigen to be administered per animal, the type of adjuvant to be used, the method of immunization and the interval between immunizations are the same as those used for preparation of polyclonal antibodies. After 1 to 30 days, preferably 2 to 5 days, from the final immunization date, animals showing antibody titers may be selected to collect antibody-producing cells. Antibody-producing cells may be exemplified by spleen cells, lymph node cells, peripheral blood cells and so on, with spleen cells or lymph node cells being preferred.

### (ii) Cell Fusion

To obtain hybridomas, cell fusion is conducted between antibody-producing cells and myeloma cells. Operations for cell fusion may be accomplished in a known manner, for example, according to the method of Kohler et al. As myeloma cells to be fused with antibody-producing cells, it is possible to use generally available established cell lines of mouse or other animal origin. Cell lines preferred for use are those having drug selectivity and having the property of not surviving in HAT selective medium (i.e., a medium containing hypoxanthine, aminopterin and thymidine) in an unfused state, but surviving only when fused with antibody-producing cells. Examples of myeloma cells include mouse myeloma cell lines (e.g., P3X63-Ag8, P3X63-Ag8U.1, SP2/O—Ag14, PAI, P3U1, NSI/1-Ag4-1, NSO/1) and rat myeloma cell lines (e.g., YB2/0), etc.

Cell fusion between the above myeloma cells and antibody-producing cells may be accomplished as follows: in a serum-free medium for animal cell culture (e.g., DMEM or RPMI-1640 medium), 1×10⁸ to 5×10⁸ antibody-producing cells may be mixed with 2×10⁷ to 10×10⁷ myeloma cells (the ratio of antibody-producing cells to myeloma cells is 10:1 to 1:1) to cause fusion reaction in the presence of a cell fusion promoter. As a cell fusion promoter, it is possible to use polyethylene glycol having an average molecular weight of 1000 to 6000 daltons or Sendai virus, etc. Alternatively, a commercially available cell fusion apparatus using electrical stimulation (e.g., electroporation) may also be used to cause cell fusion between antibody-producing cells and myeloma cells.

(iii) Screening and Cloning of Hybridomas

After cell fusion, the cells are screened to select desired hybridomas. For screening, a cell suspension may be diluted as appropriate with, e.g., RPMI-1640 medium containing 10% to 20% fetal bovine serum and then seeded by limiting dilution in microtiter plates at a density of about 0.3 cells/well by calculation, and a selective medium (e.g., HAT medium) may be added to each well, followed by culture while replacing the selective medium as appropriate. As a result, cells growing at around 10 days after the initiation of culture in the selective medium may be obtained as hybridomas.

Subsequently, the growing hybridomas are further screened. Screening of these hybridomas is not limited in any way and may be conducted in accordance with commonly used procedures. For example, aliquots of the culture supernatants contained in the wells where hybridomas have been cultured may be sampled and screened by enzyme immunoassay, radioimmunoassay, etc. More specifically, an antigen is adsorbed to 96-well plates, and the plates are then blocked with calf serum, skimmed milk, etc. The culture supernatants of hybridoma cells are reacted with the immobilized antigen at 37° C. for 1 hour and then reacted with peroxidase labeled anti-mouse IgG at 37° C. for 1 hour, followed by color development using orthophenylenediamine as a substrate. After the reaction is stopped with an acid, the plates are measured for absorbance at a wavelength of 490 nm for screening purposes. Monoclonal antibody-producing hybridomas found to be positive when measured in the above manner are cloned by limiting dilution or other techniques to thereby finally establish hybridomas which are cells producing monoclonal antibodies specifically binding to LGR6.

Cell lines (hybridomas) producing the monoclonal antibodies of the present invention may be exemplified by “Mouse-Mouse hybridoma Mu2C15” (hereinafter referred to as “Mu2C15”), “Mouse-Mouse hybridoma Mu2C21” (hereinafter referred to as “Mu2C21”) and “Mouse-Mouse hybridoma Mu2C22” (hereinafter referred to as “Mu2C22”). Upon culture of these hybridomas, homogeneous monoclonal antibodies can be prepared.

### (iv) Collection of Monoclonal Antibodies

For collection of monoclonal antibodies from the establish hybridomas, commonly used procedures, e.g., cell culture-based procedures or ascites formation procedures may be used for this purpose. In cell culture-based procedures, hybridomas are cultured in an animal cell culture medium (e.g., 10% fetal bovine serum-containing RPMI-1640 medium, MEM medium or serum-free medium) under standard culture conditions (e.g., 37° C., 5% CO₂ concentration) for 7 to 14 days, and antibodies are obtained from their culture supernatants. In the case of ascites formation procedures, the hybridomas are intraperitoneally administered at about 5×10⁶ to 2×10⁷ cells to animals of the same species as the mammal from which myeloma cells are derived, e.g., mice (BALB/c), whereby the hybridomas are allowed to grow in abundance. Then, their ascites are collected after 1 to 2 weeks. In cases where antibodies are required to be purified in the above antibody collection procedures, known techniques such as salting out with ammonium sulfate, ion exchange chromatography, gel filtration, affinity chromatography and so on may be selected as appropriate or used in combination for purification purposes.

A preferred example of the anti-LGR6 antibody of the present invention is an antibody in which the amino acid sequences of complementarity determining regions (CDRs) 1 to 3 in the heavy chain variable region (VH) comprise or consist of the amino acid sequences shown in SEQ ID NOs: 27, 29 and 31, respectively, and/or the amino acid sequences of complementarity determining regions (CDRs) 1 to 3 in the light chain variable region (VL) comprise or consist of the amino acid sequences shown in SEQ ID NOs: 33, 35 and 37, respectively. In another embodiment, a preferred example of the anti-LGR6 antibody of the present invention is an antibody in which the amino acid sequence of the heavy chain variable region (VH) comprises or consists of the amino acid sequence shown in SEQ ID NO: 23, and/or the amino acid sequence of the light chain variable region (VL) comprises or consists of the amino acid sequence shown in SEQ ID NO: 25.

### (v) Epitope for Anti-LGR6 Antibody

The epitope (antigenic determinant) for the anti-LGR6 antibody of the present invention is not limited in any way as long as it is at least a part of the antigen LGR6, but it is preferably at least a part of the extracellular region of LGR6, for example, at least a part of a region consisting of amino acids at positions 25 to 567 (SEQ ID NO: 7), a region consisting of amino acids at positions 91 to 567 (SEQ ID NO: 8), a region consisting of amino acids at positions 319 to 567 (SEQ ID NO: 9) or a region consisting of amino acids at positions 444 to 567 (SEQ ID NO: 10) in the amino acid sequence of LGR6 shown in SEQ ID NO: 6. Among them, preferred is at least a part of the region consisting of amino acids at positions 319 to 567 or the region consisting of amino acids at positions 444 to 567, and particularly preferred is at least a part of the region consisting of amino acids at positions 319 to 567. Anti-LGR6 antibody recognizing such a region (binding to such a region) is, for example, very useful for use in detection and diagnosis of cancer as described later because of ensuring high detection sensitivity for LGR6 in biological samples.

Moreover, as described above, LGR has two deletion mutants. More specifically, human LGR6 has deletion mutants such as a peptide (SEQ ID NO: 11) lacking N-terminal 43 amino acids and having different amino acids at positions 44 to 71 from those of the wild-type (SEQ ID NO: 6), a peptide (SEQ ID NO: 12) lacking N-terminal 52 amino acids and having different amino acids at positions 53 to 70 from those of the wild-type (SEQ ID NO: 6), etc.

These peptides are identical with wild-type LGR6 in a region consisting of amino acids at positions 91 to 567, a region consisting of amino acids at positions 319 to 567 or a region consisting of amino acids at positions 444 to 567 in the amino acid sequence of LGR6 shown in SEQ ID NO: 6. Thus, the antibody of the present invention allows detection of these deletion mutants.

Moreover, the epitope (antigenic determinant) for the anti-LGR6 antibody of the present invention also includes at least a part of the corresponding region in LGR6 of other animal origin.

The antibody against LGR6 of the present invention includes an antibody binding to a site (e.g., epitope) to which the antibody binds, as exemplified by an antibody binding to a site to which an antibody produced by the hybridoma of the present invention binds.

### (4) Preparation of Genetically Recombinant Antibodies

A preferred embodiment of the antibody of the present invention may be a genetically recombinant antibody. Examples of a genetically recombinant antibody include, but are not limited to, a chimeric antibody, a humanized antibody and a reconstituted human antibody, etc.

A chimeric antibody (i.e., a humanized chimeric antibody) is an antibody in which antibody variable regions of mouse origin are linked (conjugated) to constant regions of human origin (see, e.g., Proc. Natl. Acad. Sci. U.S.A. 81, 6851-6855, (1984)). For preparation of a chimeric antibody, gene recombination technology may be used for its construction such that the thus linked antibody is obtained. In this regard, the antibody variable regions of mouse origin are preferably composed of a heavy chain variable region which comprises or consists of, for example, the amino acid sequence shown in SEQ ID NO: 23 and a light chain variable region which comprises or consists of, for example, the amino acid sequence shown in SEQ ID NO: 25.

For preparation of a humanized antibody, it is possible to use a process referred to as so-called CDR grafting (CDR transplantation). CDR grafting is a technique to prepare reconstituted variable regions whose framework regions (FRs) are of human origin and whose CDRs are of mouse origin by transplanting complementarity determining regions (CDRs) from mouse antibody variable regions to human variable regions. Next, these humanized reconstituted human variable regions are linked to human constant regions. Procedures for preparation of such a humanized antibody are well known in the art (see, e.g., Nature, 321, 522-525 (1986); J. Mol. Biol., 196, 901-917 (1987); Queen C et al., Proc. Natl. Acad. Sci. USA, 86: 10029-10033 (1989); Japanese Patent No. 2828340). In this regard, the amino acid sequences of CDRs of mouse origin which can be used for the humanized anti-LGR6 antibody of the present invention are preferably, but not limited to, the amino acid sequences shown in SEQ ID NOs: 27, 29 and 31 for heavy chain variable region CDRs 1 to 3, respectively, and the amino acid sequences shown in SEQ ID NOs: 33, 35 and 37 for light chain variable region CDRs 1 to 3, respectively, by way of example.

A reconstituted human antibody (complete human antibody) is generally an antibody in which hyper variable regions serving as antigen-binding sites in its variable regions (V regions), the other regions in its V regions and its constant regions have the same structures as those of human antibody. Techniques for reconstituted human antibody preparation are also known, and in the case of gene sequences common to humans, an approach has been established for their preparation by genetic engineering procedures. Such a reconstituted human antibody may be obtained, for example, by using human antibody-producing mice which have human chromosome fragments comprising genes for human antibody heavy chain (H chain) and light chain (L chain) (see, e.g., Tomizuka, K. et al., Nature Genetics, (1977) 16, 133-143; Kuroiwa, Y. et al., Nuc. Acids Res., (1998) 26, 3447-3448; Yoshida, H. et al., Animal Cell Technology: Basic and Applied Aspects, (1999) 10, 69-73 (Kitagawa, Y., Matuda, T. and Iijima, S. eds.), Kluwer Academic Publishers; Tomizuka, K. et al., Proc. Natl. Acad. Sci. USA, (2000) 97, 722-727) or by obtaining a phage display-derived human antibody selected from human antibody libraries (see, e.g., Wormstone, I. M. et al, Investigative Ophthalmology & Visual Science., (2002) 43 (7), 2301-8; Carmen, S. et al., Briefings in Functional Genomics and Proteomics, (2002) 1 (2), 189-203; Siriwardena, D. et al., Opthalmology, (2002) 109 (3), 427-431).

Alternatively, in the present invention, a hybridoma or DNA or RNA extracted from this hybridoma may be used as a starting material to prepare a chimeric antibody, a humanized antibody or a reconstituted human antibody in accordance with the well-known approaches mentioned above.

Further, a protein fused with the antibody of the present invention may be prepared from the antibody variable regions and any other protein by known gene recombination techniques. Alternatively, such a fusion protein may be prepared by crosslinking the monoclonal antibody with any other protein using a crosslinker.

### (5) Preparation of Antibody Fragments

A fragment of the antibody against LGR6 for use in the present invention specifically binds to LGR6.

A fragment of the antibody is intended to mean a partial region of the antibody of the present invention, and examples include Fab, Fab′, F(ab′)₂, Fv, diabody (dibodies), dsFv, scFv (single chain Fv) and so on. The above antibody fragments may be obtained by cleaving the antibody of the present invention with various proteases depending on the intended purpose.

For example, Fab may be obtained by treating an antibody molecule with papain, while F(ab′)₂ may be obtained by treating an antibody molecule with pepsin. Likewise, Fab′ may be obtained by cleaving the disulfide bonds in the hinge region of the above F(ab′)₂.

In the case of scFv, cDNAs encoding antibody heavy chain variable region (H chain V region) and light chain variable region (L chain V region) may be obtained to construct DNA encoding scFv. This DNA may be inserted into an expression vector, and the resulting expression vector may be introduced into a host organism to cause expression, whereby scFv may be prepared.

In the case of diabody, cDNAs encoding antibody H chain V region and L chain V region may be obtained to construct DNA encoding scFv such that the amino acid sequence of a peptide linker has a length of 8 residues or less. This DNA may be inserted into an expression vector, and the resulting expression vector may be introduced into a host organism to cause expression, whereby diabody may be prepared.

In the case of dsFv, cDNAs encoding antibody H chain V region and L chain V region may be obtained to construct DNA encoding dsFv. This DNA may be inserted into an expression vector, and the resulting expression vector may be introduced into a host organism to cause expression, whereby dsFv may be prepared.

In the present invention, the nucleotide sequence of DNA encoding the heavy chain variable region may be exemplified by those comprising or consisting of the nucleotide sequence shown in SEQ ID NO: 22, while the nucleotide sequence of DNA encoding the light chain variable region may be exemplified by those comprising or consisting of the nucleotide sequence shown in SEQ ID NO: 24.

Moreover, specific examples of the antibody fragment of the present invention include, but are not limited to, antibody fragments comprising the amino acid sequences shown in SEQ ID NOs: 27, 29 and 31 for the amino acid sequences of VH CDRs 1 to 3, respectively, and/or comprising the amino acid sequences shown in SEQ ID NOs: 33, 35 and 37 for the amino acid sequences of VL CDRs 1 to 3, respectively. Further examples include antibody fragments comprising the amino acid sequence shown in SEQ ID NO: 23 for VH, and/or comprising the amino acid sequence shown in SEQ ID NO: 25 for VL.

A CDR-containing antibody fragment (peptide) is configured to comprise at least one or more regions of VH or VL CDRs (CDRs 1 to 3). In the case of an antibody fragment containing a plurality of CDRs, these CDRs may be linked directly or via an appropriate peptide linker. For preparation of a CDR-containing antibody fragment, DNA encoding antibody VH and VL CDRs may be constructed, and this DNA may be inserted into an expression vector for prokaryotic organisms or an expression vector for eukaryotic organisms, and the resulting expression vector may be introduced into a prokaryotic organism or a eukaryotic organism to cause expression. Alternatively, a CDR-containing peptide may also be prepared by chemical synthesis such as Fmoc method (fluorenylmethyloxycarbonyl method) and tBoc method (t-butyloxycarbonyl method).

The nucleotide sequences encoding VH CDRs 1 to 3 are preferably the nucleotide sequences shown in SEQ ID NOs: 26, 28 and 29, respectively, by way of example, while the nucleotide sequences encoding VL CDRs 1 to 3 are preferably the nucleotide sequences shown in SEQ ID NOs: 32, 34 and 36, respectively, by way of example.

### (6) Binding Affinity

The binding affinity can be determined from the binding constant (KA) and the dissociation constant (KD). The affinity equilibrium constant (K) is expressed as the KA/KD ratio. The binding affinity may be detected in the following manner.

The antibody of the present invention has a dissociation constant (KD) of at least 1×10⁻¹⁰ M and may have an affinity which is, for example, 2- to 5-fold, 5- to 10-fold, 10- to 100-fold, 100- to 1000-fold or 1000- to 10,000-fold higher than this dissociation constant. More specifically, the dissociation constant (KD) of the antibody of the present invention in relation to LGR6 binding affinity is 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵ M or 1×10⁻¹⁵ M, and is preferably 1×10⁻¹⁰ M to 1×10⁻¹³ M. Alternatively, the antibody of the present invention may have a value lower than these KD values and a higher affinity.

In this regard, if the dissociation constant (KD) of an antibody to be measured for its affinity is within about 1- to 100-fold of the KD of the antibody of the present invention, this antibody is regarded as being substantially the same as the antibody of the present invention and therefore falls within the present invention.

The binding constant (KA) and the dissociation constant (KD) may be measured by surface plasmon resonance (SPR), and any known instrument and method which allow real-time detection and monitoring of the binding rate may be used for this purpose (e.g., Biacore® T200 (GE Healthcare), ProteON XPR36 (Bio-Rad)).

### 3. Reagent for Detection or Diagnosis of Cancer (Agent for Detection or Diagnosis of Cancer)

The reagent for detection or diagnosis of cancer according to the present invention comprises the antibody or fragment thereof described above in the section “2. Antibody against LGR6 (anti-LGR6 antibody).” Since the antibody of the present invention specifically binds to LGR6 expressed in cancer, the reagent of the present invention comprising this antibody can be used for detection or diagnosis of cancer.

Examples of cancer to be detected or diagnosed in the present invention include colorectal cancer, breast cancer, uterine cancer, gastric cancer, thyroid cancer, pancreatic cancer, brain tumor, cervical cancer, esophageal cancer, tongue cancer, lung cancer, small intestinal cancer, duodenal cancer, bladder cancer, kidney cancer, liver cancer, prostate cancer, uterine cervical cancer, ovarian cancer, gallbladder cancer, pharyngeal cancer, sarcoma, melanoma, leukemia, lymphoma, multiple myeloma and so on, although preferred are cancers where LGR6 is significantly highly expressed when compared to normal cells or tissues. Examples of such cancers include colorectal cancer, breast cancer, uterine cancer, gastric cancer, thyroid cancer, pancreatic cancer and so on, with colorectal cancer, breast cancer, uterine cancer and gastric cancer being preferred, and with colorectal cancer and breast cancer being more preferred.

The reagent of the present invention may further comprise a pharmaceutically acceptable carrier, in addition to the antibody of the present invention. The term “pharmaceutically acceptable carrier” refers to any type of carrier (e.g., liposomes, lipid vesicles, micelles), diluent, excipient, wetting agent, buffering agent, suspending agent, lubricant, adjuvant, emulsifier, disintegrant, absorbent, preservative, surfactant, coloring agent, flavoring agent or the like, which is suitable for the reagent of the present invention.

In another embodiment, the present invention provides the use of a monoclonal antibody against LGR6 or a fragment thereof for the manufacture of a reagent for detection or diagnosis of cancer. Moreover, the present invention also provides a monoclonal antibody against LGR6 or a fragment thereof for use in the detection or diagnosis of cancer. Further, the present invention provides a monoclonal antibody against LGR6 or a fragment thereof for use as a reagent for detection or diagnosis of cancer. Such a monoclonal antibody against LGR6 or a fragment thereof is as described above in the section “2. Antibody against LGR6 (anti-LGR6 antibody).”

### 4. Method for Detection or Diagnosis of Cancer

The method for detection or diagnosis of cancer according to the present invention comprises the step of contacting the antibody or fragment thereof described above in the section “2. Antibody against LGR6 (anti-LGR6 antibody)” with a biological sample taken from a subject (hereinafter also simply referred to as a “biological sample”) and detecting LGR6 in the sample. In this case, it is preferred that the detection results of LGR6 are connected with the possibility of cancer.

In the context of the present invention, the term “subject” is intended to include, for example, humans, rabbits, guinea pigs, rats, mice, hamsters, cats, dogs, goats, pigs, sheep, cows, horses, monkeys and other mammals, with humans being preferred.

Likewise, the biological sample is not limited in any way and refers to, for example, mammalian tissue or cells, a homogenate or extract thereof, body fluid or excrement per se or a sample containing any of them. Moreover, the biological sample also includes samples obtained from mammalian tissue or cells, a homogenate or extract thereof, body fluid and excrement through any type of treatment, e.g., dilution, separation, nucleic acid or protein extraction, protein denaturation, etc. A biological sample preferred for screening of colorectal cancer or gastric cancer is body fluid or excrement, particularly feces. For screening of bladder cancer or kidney cancer, it is possible to use tissue or urine. Likewise, for screening of breast cancer or uterine cancer, it is possible to use mother's milk, sanitary goods or the like. Any biological samples may be applied by being pretreated appropriately.

When feces is used as a sample, the sample is preferably prepared by the method described in JP 2005-46065 A. This method is designed to efficiently collect cancer cells from feces. More specifically, feces and a buffer are introduced into a stomacher bag to prepare a suspension of feces. This suspension is filtered through a multi-filter apparatus to capture cells on the final filter whose bore size is set to 10 μm or less, from which the cells are then collected using Ber-EP4 antibody-bound magnetic beads (Dynabeads Epithelial Enrich, Dynal Biotech). In addition to this, efficient cell collection techniques (e.g., Percoll centrifugation) commonly used by those skilled in the art may be used for this purpose.

In various cancers including colorectal cancer and breast cancer, any tumor marker has not yet been established which allows early detection using body fluid. Commonly used markers may show false negative results even in advanced stages of cancer. In contrast, when using the antibody of the present invention, a trace amount of cells, which express LGR6 serving as a marker, contained in body fluid or excrement can be detected and/or measured before cancer has progressed.

If the “subject” is a human, the subject includes cancer patients (e.g., early cancer patients, advanced cancer patients) and normal subjects. For example, in the case of colorectal cancer, gastric cancer or the like, early cancer refers to a state where cancer cells remain in the mucosa or submucosa, while advanced cancer refers to a state where cancer cells have reached the muscularis propria or deeper layers. Based on these criteria, patients may be categorized and provided for the detection of the present invention. Moreover, as a blood sample, preferred is peripheral blood and more preferred is peripheral blood-derived serum. Further, tissues include cancer tissues and normal tissues which may develop cancer.

In the context of the present invention, the term “contact” is intended to mean that the antibody of the present invention and a biological sample taken from a subject are allowed to exist in the same reaction system, as exemplified by mixing the antibody of the present invention and the biological sample in a reaction well, adding the antibody of the present invention to the biological sample, adding either the antibody of the present invention or the biological sample to a carrier on which the other is immobilized, etc.

In the present invention, for LGR6 detection or for measurement or evaluation of LGR6 expression levels, it is possible to use, for example, enzyme immunoassay (EIA), fluorescence immunoassay (FIA), radioimmunoassay (RIA), chemiluminescence immunoassay (CIA), turbidimetric immunoassay, immunonephelometry, latex agglutination, latex turbidimetry, hemagglutination reaction, particle agglutination reaction, Western blotting, immunostaining, immunoprecipitation, immunochromatography and so on.

Any device may be used for such detection and other purposes, and examples include a micro-well plate, an array, a chip, a flow cytometer, a surface plasmon resonance apparatus, an immunochromatographic strip and so on. In the case of using such a device, signals (e.g., color development, fluorescence intensity, luminescence intensity) can be detected, measured and/or evaluated, and the results of detection, measurement or evaluation can be connected with the possibility of cancer, etc.

In the present invention, based on the results of LGR6 detection or on the results of measurement or evaluation of LGR6 expression levels, a subject can be diagnosed for the presence or absence of the possibility of suffering from cancer.

In cases where the detection or diagnostic method of the present invention is accomplished by enzyme immunoassay or fluorescence immunoassay, an appropriately pretreated sample as described above (e.g., cells including cancer cells collected from feces or sections prepared from tissue taken from a patient) may be immobilized on a solid phase (e.g., micro-well plates or slide glasses) and provided for immunological reaction. Alternatively, a cell suspension may be reacted with the antibody in a tube. Moreover, it is also possible to use procedures in which the monoclonal antibody of the present invention is immobilized on beads or micro-well plates and then reacted with cells.

In this case, the monoclonal antibody of the present invention may be labeled with an enzyme or a fluorescent substance to thereby directly detect the reaction as fluorescence signals, or alternatively, a labeled secondary antibody which binds to the monoclonal antibody of the present invention may be used to indirectly detect signals. As a labelling substance, any substance commonly used by those skilled in the art may be used, as exemplified by peroxidase (POD), alkaline phosphatase, β-galactosidase, a biotin-avidin complex, etc. Likewise, the detection method used for this purpose may be any of competitive assay, sandwich assay or direct adsorption assay, etc. Moreover, the strength of the reaction or the ratio of cells bound to the antibody among all cells in the reacted sample may be measured and compared with the predetermined reference value or index to thereby determine or diagnose the possibility of suffering from cancer.

In the present invention, when LGR6 detection or measurement or evaluation of LGR6 expression levels is accomplished by using immunostaining, for example, the degree of staining in immunohistological staining (i.e., the detection results of LGR6) may be ranked as strongly positive (Strong), positive (Moderate), weakly positive (Weak), negative (Negative), etc., and the degree of staining is connected with the possibility of cancer to thereby detect cancer or diagnose a subject for the presence or absence of the possibility of cancer (the possibility of suffering from cancer).

Moreover, under the assumption that the extracellular region of LGR6 is released into blood, the expression level of LGR6 in a blood sample may be measured to thereby detect cancer or diagnose a subject for the presence or absence of the possibility of cancer. In this case, for detection of cancer by connecting the results measured for the expression level of LGR6 with the possibility of cancer, it is desired to define a critical value (cut-off value) for the expression level of LGR6 in a biological sample.

The cut-off value for the expression level of LGR6 may be determined as follows, by way of example. First, biological samples derived from cancer patients are measured for their expression levels of LGR6. The number of patients intended here is two or more, for example, 5 or more, 10 or more, 50 or more, or 100 or more. Preferably, biological samples derived from two or more normal subjects have also been measured for their expression levels of LGR6, and the number of normal subjects intended here is two or more, for example, 5 or more, 10 or more, 50 or more, or 100 or more. Then, from all the cases including both the group of biological samples derived from cancer patients and the group of biological samples derived from normal subjects, the cut-off value for the expression level of LGR6 is determined by statistical processing. For statistical processing, 4-parameter logistic fitting may be used to prepare a standard curve. On the basis of the signal value in a LGR6-free sample, the double of this numerical value may be defined to be the cut-off value. Cases provided for statistical analysis may also be classified by the type of cancer (e.g., colorectal cancer, breast cancer, uterine cancer, gastric cancer, thyroid cancer, pancreatic cancer, leukemia), the stage of cancer, the presence or absence of recurrence, the presence or absence of metastasis, before or after surgery, etc. Furthermore, statistical processing may also be accomplished by combining, as appropriate, the measured values of LGR6 expression levels in normal subjects and the measured values of LGR6 expression levels in cancer patients when classified by the type of cancer, the stage of cancer, the presence or absence of recurrence, the presence or absence of metastasis, before or after surgery, etc.

In the present invention, the critical value (cut-off value) for the expression levels of LGR6 (extracellular region) in blood samples is, for example, about 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 26 ng/ml, 27 ng/ml, 28 ng/ml, 29 ng/ml, 30 ng/ml, 35 ng/ml, 40 ng/ml, 45 ng/ml, 50 ng/ml, 55 ng/ml, 60 ng/ml or 65 ng/ml when expressed as a concentration in serum, with about 20 ng/ml being preferred.

When a sample is measured for its expression level of LGR6 under the above conditions, if the measured expression level of LGR6 is equal to or higher than the above cut-off value, it is possible to make a determination that cancer was detected or to make a diagnosis that the subject has the possibility of suffering from cancer. In the present invention, an upper limit may be provided for the value of serum concentration required for the above determination or diagnosis. For example, if the measured value is equal to or higher than the above cut-off value and is equal to or lower than the given upper limit value (e.g., 200 ng/ml or less, 190 ng/ml or less, 180 ng/ml or less, 170 ng/ml or less, 160 ng/ml or less, 150 ng/ml or less, 145 ng/ml or less, 140 ng/ml or less, 135 ng/ml or less, 130 ng/ml or less), it is possible to detect cancer or diagnose a subject for the presence or absence of the possibility of cancer.

In the present invention, the probability of the detection results obtained when cancer was detected is 60% or more, 70% or more, 80% or more, 90% or more, or 95% or more, and preferably 99% or more.

Further, in addition to the embodiment where the expression level of LGR6 measured in a sample from a single subject (patient) is compared with the above cut-off value to detect or diagnose the relation to cancer, biological samples derived from a plurality of patients may be used and measured for their expression levels of LGR6 in another embodiment of the present invention. Thus, in the same manner as used to determine the above cut-off value, in a population (primary population) including a given number of normal subjects and patients, their expression levels of LGR6 may be measured and processed by statistical analysis, whereby these subjects belonging to the population may be divided into a group where cancer was detected and a group where cancer was not detected. Further, the thus obtained measured values are used as master data, and a comparison may be made between these master data and the expression levels of LGR6 in samples derived from individual subjects to be detected or diagnosed in another population (secondary population).

Alternatively, the data of the respective patients may be incorporated into the values of the above population and subjected again to data processing of LGR6 expression levels to increase the number of cases of target patients (population). The increased number of cases can improve the accuracy of the critical value for the expression level of LGR6 to thereby improve the accuracy of detection or diagnosis in a single subject or a plurality of subjects.

In the present invention, a comparison may also be made between (i) the expression level of LGR6 in a biological sample from a subject and (ii) the expression level of LGR6 in a biological sample from a normal subject.

Moreover, if the expression level of LGR6 in (i) above is higher than the expression level of LGR6 in (ii) above, e.g., is about 10% or more, about 20% or more, about 30% or more, about 40% or more, about 50% or more, about 60% or more, about 70% or more, about 80% or more, about 90% or more, or about 100% or more higher than the expression level of LGR6 in a biological sample from a normal subject, it is possible to make a determination that cancer was detected or to make a diagnosis that the subject has the possibility of suffering from cancer.

The above detection results may be used, for example, as main data or supplemental data in screening of cancer or in definitive diagnosis of therapeutic effects.

Since LGR6 expression is observed in patients with various types of cancer, it cannot be identified in some cases what type of cancer a subject has even when the expression level of LGR6 is detected during medical examination, etc. For this reason, such a subject is evaluated as being “suspected to have cancer” in group health screening or the like, and this evaluation result may be used later as supplemental data to identify the type of cancer and/or determine the degree of cancer progression (for detailed examination). Moreover, when LGR6 was detected by using a sample derived from a patient who was suspected to have some type of cancer, such a result may be used as main data on cancer type (e.g., definitive diagnosis). It should be noted that identification of cancer type may be accomplished by using other tumor markers, diagnostic imaging, pathological diagnosis, etc.

Namely, the present invention also provides a method for aiding detection or diagnosis of cancer, which comprises the step of contacting a monoclonal antibody against LGR6 or a fragment thereof with a biological sample taken from a subject and detecting LGR6 in the sample.

### 5. Kit for Detection or Diagnosis of Cancer

The anti-LGR6 antibody of the present invention may be provided in the form of a kit for detection or diagnosis of cancer. The kit of the present invention comprises the antibody and may further comprise a labelling substance, or alternatively, may comprise an immobilized reagent in which the antibody or the antibody labeled with the labelling substance is immobilized. The antibody labeled with the labelling substance is intended to mean the antibody labeled with an enzyme, a radioisotope, a fluorescent compound, a chemiluminescent compound or the like. In addition to the above constituent elements, the kit of the present invention may further comprise other reagents required to accomplish the detection of the present invention, as exemplified by an enzyme and a substrate (e.g., a chromogenic substrate), a substrate diluent, an enzyme reaction stop solution, or an analyte diluent and so on when the labeled product is an enzymatically labeled product. Moreover, the kit of the present invention may also comprise various buffers, sterilized water, various cell culture vessels, various reaction vessels (e.g., Eppendorf tubes), a blocking agent (e.g., bovine serum albumin (BSA), skimmed milk, goat serum or other serum components), a detergent, a surfactant, various plates, an antiseptic (e.g., sodium azide), an instruction manual for experimental operations (manufacturer's instructions) and so on. These reagents are provided, e.g., in an immobilized state, in an aqueous solution state or in a lyophilized state, and may be reconstituted into an appropriate state before use.

The kit of the present invention can be used effectively to accomplish the above detection method of the present invention and is extremely useful. The use of the kit of the present invention allows highly accurate screening of cancer patients during medical examination or the like, early diagnosis of cancer, determination of the degree of cancer progression, monitoring of therapeutic effects during treatment, and acquisition of information useful to predict metastasis and/or recurrence, so that the mortality due to cancer can be reduced.

Details on the anti-LGR antibody, the type of cancer, the detection method and so on are the same as described above.

### EXAMPLES

The present invention will be further described in more detail by way of the following illustrative examples, which are not intended to limit the scope of the invention.

### Example 1

### Preparation of Anti-LGR6 Monoclonal Antibodies

### (1) Cell Culture

Mouse myeloma cell line P3X63-Ag8 (ATCC Accession No. CRL-1580), human colorectal cancer cell line HT-29 (ATCC Accession No. HTB38), human fetal kidney 293T cells were each cultured and subcultured in RPMI 1640 medium (Sigma) containing 10% (v/v) serum (Hyclone) at 37° C. under 5% CO₂ for 48 to 72 hours, such that the confluency did not exceed 80%.

### (2) Cloning of LGR6 Gene

293T cells were cultured and their total RNA was extracted with a Qiagen RNeasy Mini kit. From the extracted total RNA (2 μg), cDNA was synthesized by reverse transcription (RT) reaction at 50° C. for 1 hour with SuperScript III reverse transcriptase (Invitrogen), followed by heating at 85° C. for 5 minutes to stop the reaction. The resulting cDNA was used as a template for PCR reaction with the following primers and with KOD PLUS (TOYOBO) to thereby amplify DNA comprising a nucleotide sequence (SEQ ID NO: 13) encoding amino acids at positions 319 to 567 of LGR6.

### Primer Sequences

Forward:
(SEQ ID NO: 14)
TTTGAATTCGATGCAGGAGTTTCCAGATCTCAAAGGC
Reverse:
(SEQ ID NO: 15)
TTTGCGGCCGCCACGTGTGAAGCAAAGGCCAAG

The PCR reaction was conducted by preincubation at 95° C. for 10 minutes and subsequent 40 cycles of denaturation at 95° C. for 15 seconds and annealing/elongation at 58° C. for 1 minute to thereby amplify the desired gene fragment.

The resulting amplified fragment was cleaved with restriction enzymes (EcoRI and NotI) at the restriction enzyme sites located on the primers and then integrated into pET32b vector (Invitrogen) to thereby obtain DNA comprising a nucleotide sequence encoding a partial protein of LGR6 having Trx-, S- and His-tags on the N-terminal side and a His-tag on the C-terminal side.

### (3) Preparation of LGR6 Partial Protein

BL21(DE3) (Invitrogen) was transformed with the vector prepared in (2) above and cultured in LB medium [1% (w/v) tryptone (Sigma), 0.5% (w/v) yeast extract (Sigma), 0.5% (w/v) NaCl (Sigma)] supplemented with 1% (w/v) glucose. After the medium turbidity reached 0.6 at a wavelength of 600 nm, 1 mM IPTG (WAKO) was added and culture was continued for 16 hours. The microbial cells were collected by centrifugation and then homogenized by ultrasonication to obtain a fraction containing the extracellular region of LGR6 as an insoluble protein.

About 10 mg of the sample was dissolved in Buffer A (1 M guanidine hydrochloride (Sigma), 10 mM DTT (Sigma), 10 mM EDTA (Sigma)) and reacted at 37° C. for 1 hour. The reaction solution was added gently to 1 L of Buffer B (50 mM Tris, 150 mM NaCl, 5% glycerol, 0.4 mM oxidized glutathione (Sigma), pH 8.5) and stirred at 4° C. for 18 hours. The dissolved sample was applied to a Ni-sepharose column (GE) and eluted with Buffer C (50 mM potassium phosphate buffer, 150 mM NaCl, 200 mM imidazole, pH 8.0), followed by dialysis against imidazole-free Buffer C to obtain a partial protein of LGR6 including its hinge region in purified form. This protein is hereinafter referred to as ECD2. The amino acid sequence of ECD2 is shown in SEQ ID NO: 9.

### (4) Immunization

ECD2 prepared above was mixed with an equal amount of Freund's complete adjuvant and intraperitoneally administered to BALB/c mice in a volume of 100 μl (about 40 μg/mouse). After about 3 weeks, ECD2 was also mixed with an equal amount of Freund's incomplete adjuvant and intraperitoneally administered to the mice. This operation was repeated 7 to 12 times, followed by confirmation of an increase in antibody titers. The mice showing increased titers were intravenously administered with ECD2 (about 40 μg) as the final immunization, and after 3 days, their spleens were excised.

### (5) Cell Fusion

Mouse spleen lymphocytes were electrically fused with mouse myeloma cell line P3X63-Ag8. For cell fusion, 1×10⁸ spleen cells were mixed with 0.25×10⁸ cells of the myeloma cell line and suspended in EP Buffer (0.3 M mannitol, 0.1 mM CaCl₂, 0.1 mM MgCl₂) to give a cell density of 0.25×10⁸ cells/mL, followed by cell fusion with an electro cell fusion generator LF201 (Nepa Gene Co., Ltd., Japan). Fusion conditions were set in accordance with the manufacturer's recommended protocols.

The fused cells were suspended in HAT medium (Invitrogen) and dispensed into thirty 96-well plates in a volume of 100 μL per well. During culture, 200 μL of HAT medium was added to each well. After culture for 11 to 16 days, the plates were observed under a microscope, indicating that 5 to 12 colonies were formed per well.

### (6) ELISA

Culture supernatants were collected from wells where hybridomas were growing, and screened to select hybridomas producing monoclonal antibodies reactive to ECD2. ECD2 was diluted with PBS(−) and dispensed into 96-well ELISA plates (Nunc) in an amount of 1 μg per well, and immobilized on the plate surface by being allowed to stand overnight at 4° C. Then, after the plates were washed three times with 350 μL of PBS(−) containing 0.05% Tween 20 (hereinafter abbreviated as PBS-T), PBS-T containing 1% skimmed milk was dispensed in a volume of 300 μL per well, followed by blocking for 1 hour at room temperature. After washing with PBS-T, the culture supernatants of hybridomas were dispensed into the ECD2-immobilized ELISA plates and reacted for 1 hour at room temperature. After washing with PBS-T, a 10000-fold dilution of peroxidase (hereinafter abbreviated as POD)-labeled anti-mouse immunoglobulin antibody (BETHYL) was dispensed in a volume of 100 μL per well and reacted for 1 hour at room temperature. After washing in the same manner, a POD substrate prepared at 1 mg/mL POD was added to cause color development at room temperature for 5 minutes. After the reaction was stopped with 1.5 N sulfuric acid, the plates were measured for absorbance at 490 nm with a plate reader (Molecular Devices).

### (7) Obtaining of Monoclonal Antibodies

From among antibodies produced from the hybridoma cells formed in (5) above, antibodies reactive to ECD2 were selected in the same manner as shown in (6) above. Moreover, to remove antibodies reactive to the Trx-, S- and His-tags added during ECD2 preparation, pET32b vector alone was introduced into E. coli and the same procedure as shown in (3) above was repeated to express and purify a protein for use as a negative control. Hereinafter, this protein for use as a negative control is referred to as Tag.

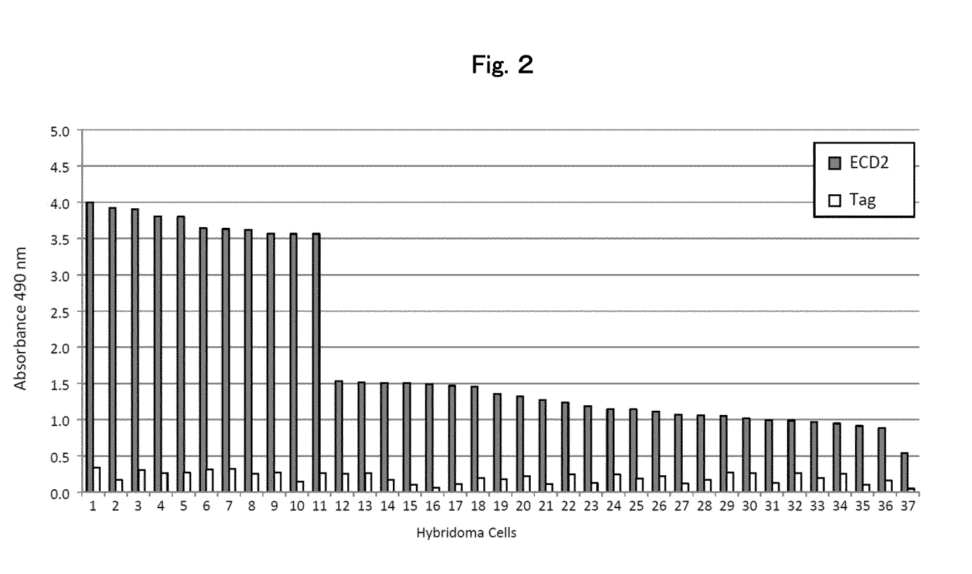

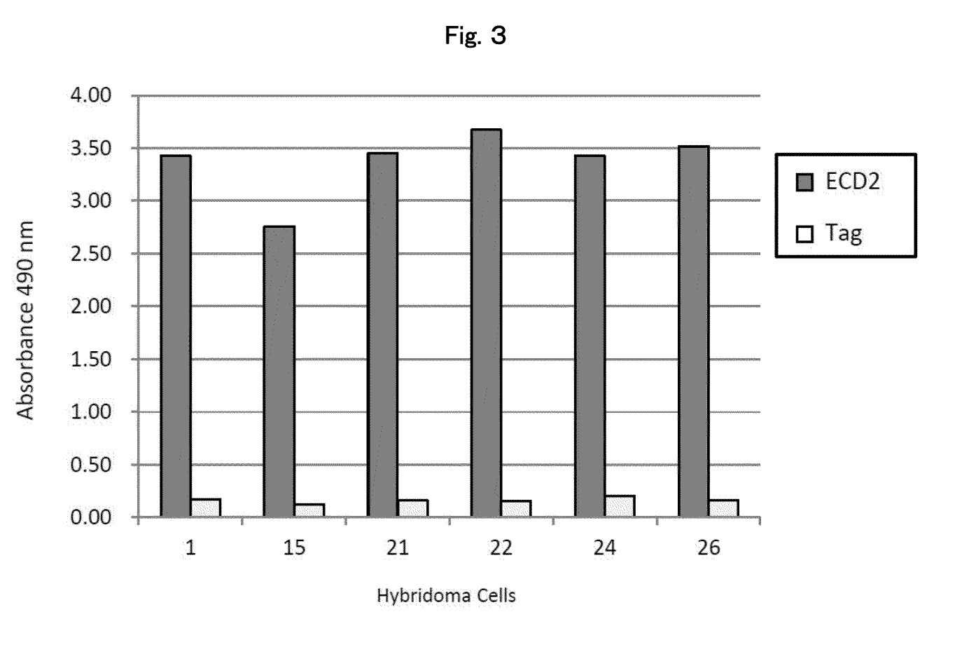

As a result of selecting hybridoma cells producing monoclonal antibodies which were reactive to ECD2 and not reactive to Tag, 37 clones were obtained. The results of ELISA for individual antibodies are shown in FIG. 2. From among these hybridoma cells, those producing antibodies which were strongly reactive to ECD2 and not reactive to Tag were selected and cloned singly by limiting dilution, whereby 6 clones of hybridoma cells were established. The reactivity of antibodies produced from these hybridomas was measured by ELISA and the results obtained are shown in FIG. 3. Hereinafter, antibodies from the 6 clones are also referred to as “Mu2C1,” “Mu2C15,” “Mu2C21,” “Mu2C22,” “Mu2C24” and “Mu2C26,” respectively.

These hybridoma cells were each cultured in ten 10 cm dishes to reach 90% confluency and cultured for 10 days in a 1:1 mixed medium of HT medium (Invitrogen) and EX CELL Sp2/0 (Nichirei Bioscience Inc., Japan). The culture supernatants were collected and purified with a Protein G column. The Protein G column (GE Healthcare) was used in a volume of 0.5 mL relative to 100 mL culture supernatant. The cultured solutions were each passed at a flow rate of 1 to 3 ml/min through the Protein G column which had been equilibrated with PBS, followed by washing with 6 mL of a washing buffer (25 mM Tris-HCl (pH 7.4), 140 mM NaCl, 10 mM KCl). Then, antibody proteins were eluted with 1 mL of an elution buffer (0.1 M glycine (pH 2.5)) and neutralized with 3 M Tris-HCl (pH 7.4) to be within pH 7.0 to 7.4. The antibodies were concentrated with Amicon Ultra 30 (Millipore) and the buffer was replaced with PBS.

### Example 2

### Analysis of Anti-LGR6 Monoclonal Antibodies

### (1) Western Blotting

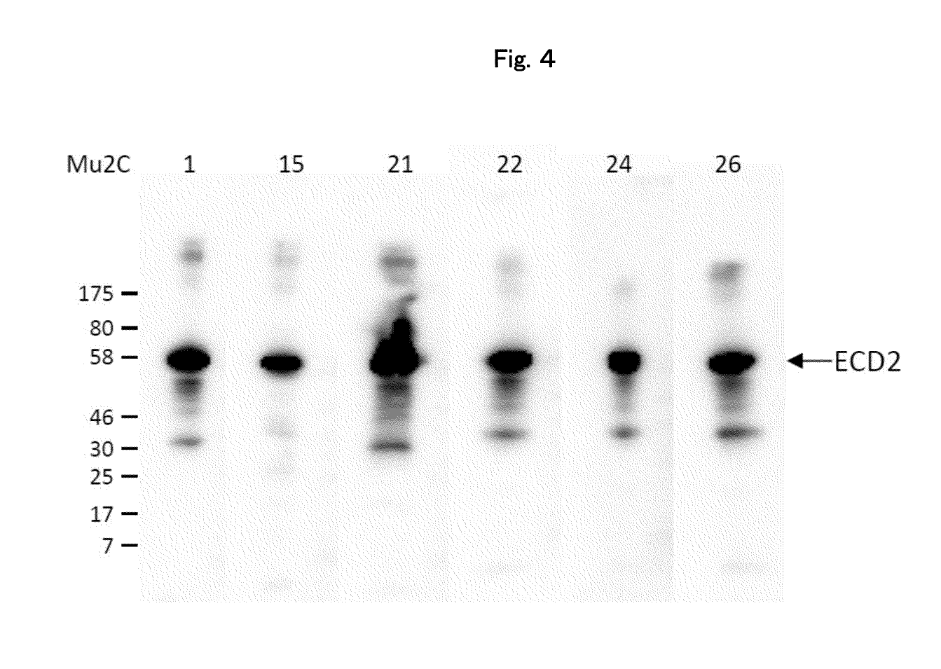

The antibodies obtained in Example 1. (7), which were produced by the six lines of hybridoma cells “Mu2C1,” “Mu2C15,” “Mu2C21,” “Mu2C22,” “Mu2C24” and “Mu2C26” were analyzed by Western blotting. First, these antibodies were analyzed for their reactivity to the antigen ECD2. ECD2 prepared in Example 1. (3) was measured for its protein concentration by the Bradford assay, and then adjusted to 1 μg/mL and provided for SDS-PAGE. After electrophoresis, the protein was transferred onto a PVDF membrane (PIERCE) with a Trans-Blot SD cell (BioRad Laboratories) in accordance with the manufacturer's recommended protocols. Skimmed milk dissolved at a concentration of 5% (w/v) in TBS-T was used to block the membrane at room temperature for 30 minutes, and the membrane was washed twice with TBS-T. The six types of monoclonal antibodies prepared in Example 1. (7) were each diluted to 1 μg/mL with TBS-T and reacted with the membrane for 1 hour at room temperature. After washing three times with TBS-T, anti-mouse IgG polyclonal antibody-HRP label (BETHYL) was diluted 10,000-fold with TBS-T for use as a secondary antibody. This dilution was reacted with the membrane at room temperature for 30 minutes, followed by washing three times with TBS-T. The membrane was soaked in Immobilon (Millipore) and then wrapped, followed by signal detection with LAS-3000 (Fuji Photo Film Co., Ltd., Japan). The experimental results obtained are shown in FIG. 4. These results indicated that the six types of antibodies were also reactive to denatured ECD2.

### (2) Immunocytological Staining

The six types of antibodies obtained in Example 1. (7) were analyzed for their reactivity to colorectal cancer cells (HT-29). Hereinafter, the antibodies produced by the hybridoma cells are also expressed as their respective clone numbers.

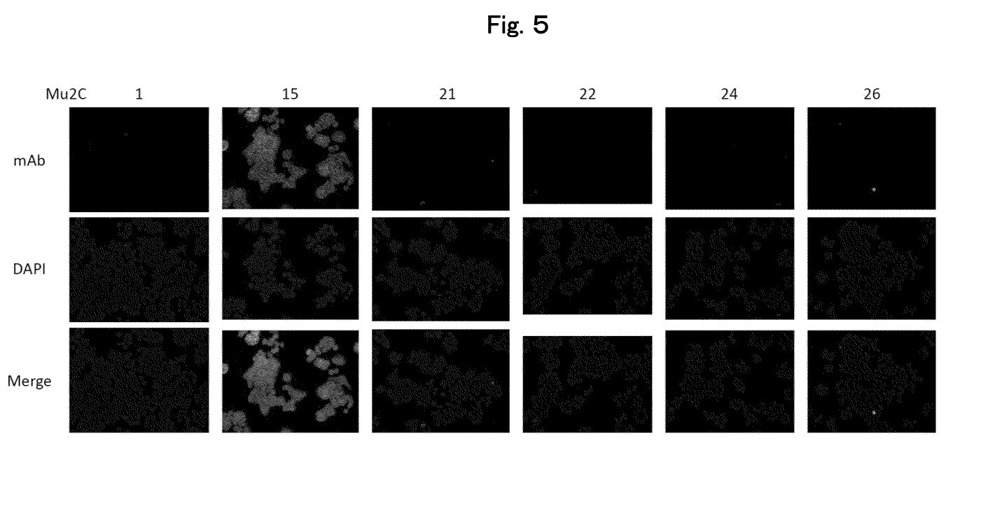

HT-29 cells were cultured to reach 80% confluency and then seeded onto cover slips coated with Cellmatrix Type I-A (Nitta Gelatin Inc., Japan). After culture for 2 days, the cells were fixed with 10% neutral buffered formalin (WAKO). After being treated with 0.3% (v/v) aqueous hydrogen peroxide for 20 minutes, the cover slips were washed three times with TBS-T and treated with TBS-T containing 5% (w/v) skimmed milk, followed by addition of the antibodies purified in (7) above at a concentration of 10 μg/mL and reaction at 4° C. for 16 hours. After washing three times with TBS-T, anti-mouse IgG polyclonal antibody-Alexa Fluor 488 label or anti-mouse IgM polyclonal antibody-Alexa Fluor 488 label was reacted as a secondary antibody at room temperature for 30 minutes. After washing three times with TBS-T, the cover slips were sealed with Mounting Medium (Vector Shield). FIG. 5 shows the results analyzed for fluorescence intensity under the same conditions. Strong signals were observed only in Mu2C15, thus suggesting that the cell membrane and cytoplasm were stained. Namely, Mu2C15 was confirmed to recognize LGR6 on the cell surface. Further, since LGR6 is a membrane protein with seven transmembrane domains, signals observed in the cytoplasm are indicative of the finding that LGR6 on the cell membrane was taken up into cells through internalization. A phenomenon where LGR6 causes internalization has not so far been reported, and hence this is the first report of this phenomenon.

### (3) FACS

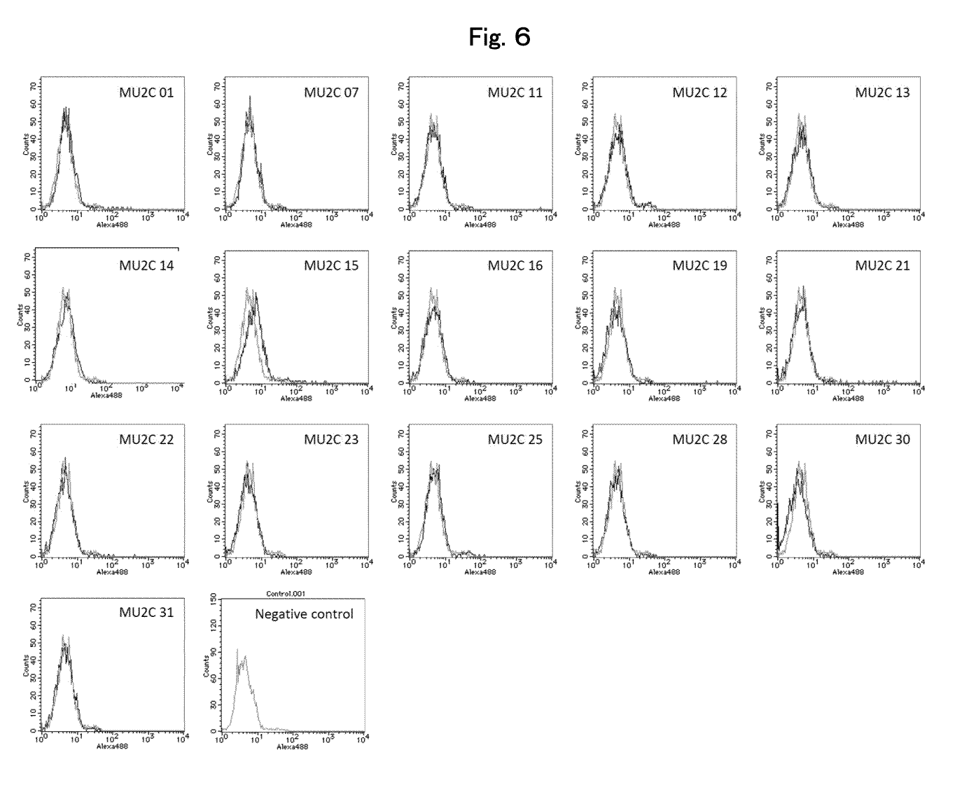

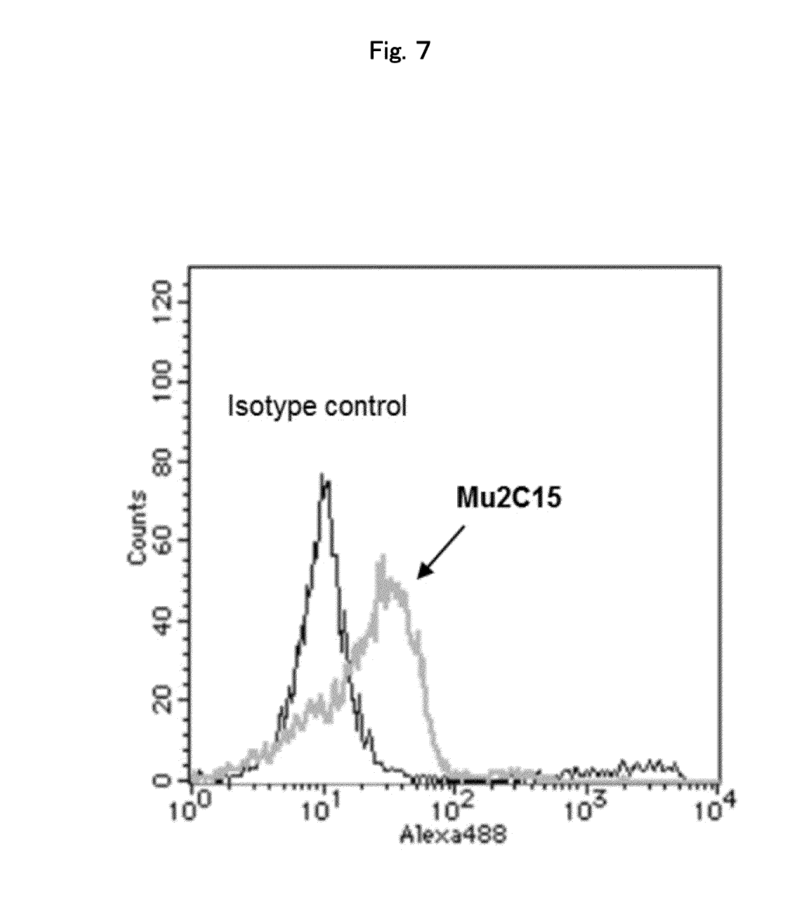

HT-29 cells, which are human colorectal cancer cells, were cultured to reach 90% confluency. After washing twice with PBS, the cells were detached non-enzymatically from the plates with Cell Dissociation Buffer (Invitrogen) and collected into 1.5 mL tubes. From 16 lines among the hybridoma cells obtained in Example 1. (7), i.e., Mu2C1, 7, 11, 12, 13, 14, 15, 16, 19, 21, 22, 23, 25, 28, 30 and 31, culture supernatants were each collected and added in a volume of 100 μL, and then reacted for 60 minutes. As a control for comparison purposes, an antibody-free medium was prepared and provided for reaction. After washing twice with PBS+2% FBS, Alexa Fluor 488-labeled goat anti-mouse IgG (Invitrogen) was added as a 1/1000 dilution in PBS+2% FBS, and then reacted for 30 minutes. After washing twice with PBS+2% FBS, the cells were analyzed by Guava Flow Cytometry (Millipore). The results obtained are shown in FIG. 6. The antibodies except for Mu2C15 and Mu2C30 showed signals similar to those of the negative control and did not react with LGR6 in its native structure, whereas Mu2C15 and Mu2C30 were found to show slight changes in signals. Moreover, in the case of Mu2C15, the antibody was purified to increase the antibody concentration to 100 μg/mL and provided for reaction with SW480 cells. The results obtained are shown in FIG. 7. In this case, a clear shift in signals was observed when compared to the negative control. Namely, the antibody was found to react with living cells (living cancer cells).

These results indicated that the antibodies of the present invention were useful for detection of living cancer cells present in biological samples, e.g., blood samples.

### (4) Determination of Nucleotide Sequences and Amino Acid Sequences for Antibody Variable Regions

The hybridoma cells obtained in Example 1. (7) were cultured and their total RNA was extracted with a Qiagen RNeasy Mini kit. From the extracted total RNA (500 ng), cDNA was synthesized by reverse transcription (RT) reaction at 42° C. for 1.5 hours with a SMARTer RACE cDNA Amplification Kit (Clontech), followed by heating at 70° C. for 10 minutes to stop the reaction. The resulting cDNA was used as a template for PCR reaction with the following primers and with KOD PLUS ver.2 (TOYOBO) to thereby amplify a desired gene. As a representative example of the antibodies produced from the hybridoma cells obtained in Example 1. (7), the antibody produced by hybridoma cells “Mu2C15” (also referred to as “Mu2C15” or “Mu2C15 antibody”) was determined for amino acid sequences of its variable regions in the following manner.

To amplify an H chain variable region fragment of the antibody, the following primers (SEQ ID NOs: 16, 17 and 18) were used in PCR reaction (30 cycles of denaturation at 94° C. for 2 minutes, subsequent annealing at 56° C. for 15 seconds and elongation at 68° C. for 45 seconds) to thereby obtain the desired fragment.

Long:
(SEQ ID NO: 16)
5′-CTAATACGACTCACTATAGGGCAAGCAGTGGTAT
CAACGCAGAGT-3′
Short:
(SEQ ID NO: 17)
5′-CTAATACGACTCACTATAGGGC-3′
mIgG1_CH_reverse:
(SEQ ID NO: 18)
5′-CCAGGGGCCAGTGGATAGACAGATGGGGG
TGTCG-3′

To amplify an L chain leader sequence and an L chain variable region fragment of the antibody, the above primers (Long and Short) and the following primer (SEQ ID NO: 19) were used in PCR reaction (30 cycles of denaturation at 94° C. for 2 minutes, subsequent annealing at 56° C. for 15 seconds and elongation at 68° C. for 45 seconds) to thereby obtain the desired fragment.

mIgkappa_R3:
(SEQ ID NO: 19)
5′-GCACCTCCAGATGTTAACTGCTCACT-3′

The purified PCR amplification fragments were each mixed with 10 mM dNTP Mix (Invitrogen) and 2× GoTaq Maxter Mix (Promega) and reacted at 70° C. for 15 minutes, followed by ice cooling at 4° C. for 2 minutes to ensure dA addition to the 3′-terminal end. Then, using a pGEM-T-Easy Vector System (Promega), the H chain fragment and the L chain fragment were cloned by the so-called TA cloning method. Then, the following primers (SEQ ID NO: 20 and SEQ ID NO: 21) were used in sequencing reaction to determine the sequences of the H chain and L chain variable regions.

T7 promoter primer v2:
(SEQ ID NO: 20)
GTCACGACGTTGTAAAACGA
SP6 promoter primer:
(SEQ ID NO: 21)
ATTTAGGTGACACTATAGAA

The amino acid sequence of the H chain variable region (hereinafter also referred to as “VH”) in the Mu2C15 antibody is shown in SEQ ID NO: 23, and the nucleotide sequence encoding this region is shown in SEQ ID NO: 22. Likewise, the amino acid sequence of the L chain variable region (hereinafter also referred to as “VL”) in the Mu2C15 antibody is shown in SEQ ID NO: 25, and the nucleotide sequence encoding this region is shown in SEQ ID NO: 24.

Moreover, in the amino acid sequences of the variable regions in the Mu2C15 antibody, regions showing particularly large changes (i.e., having low homology) when compared to the amino acid sequences of the variable regions in the existing anti-LGR6 antibodies were identified as complementarity determining regions (CDRs) of this antibody.

The amino acid sequences of the identified CDRs in the Mu2C15 antibody are shown below.

VH CDR1:
(SEQ ID NO: 27)
GYTFTTYT
VH CDR2:
(SEQ ID NO: 29)
INPNNGYT
VH CDR3:
(SEQ ID NO: 31)
ARRDYYRFSTGFAY
VL CDR1:
(SEQ ID NO: 33)
QSLVHTNGNTY
VL CDR2:
(SEQ ID NO: 35)
KVS
VH CDR3:
(SEQ ID NO: 37)
SQSTHVPYT

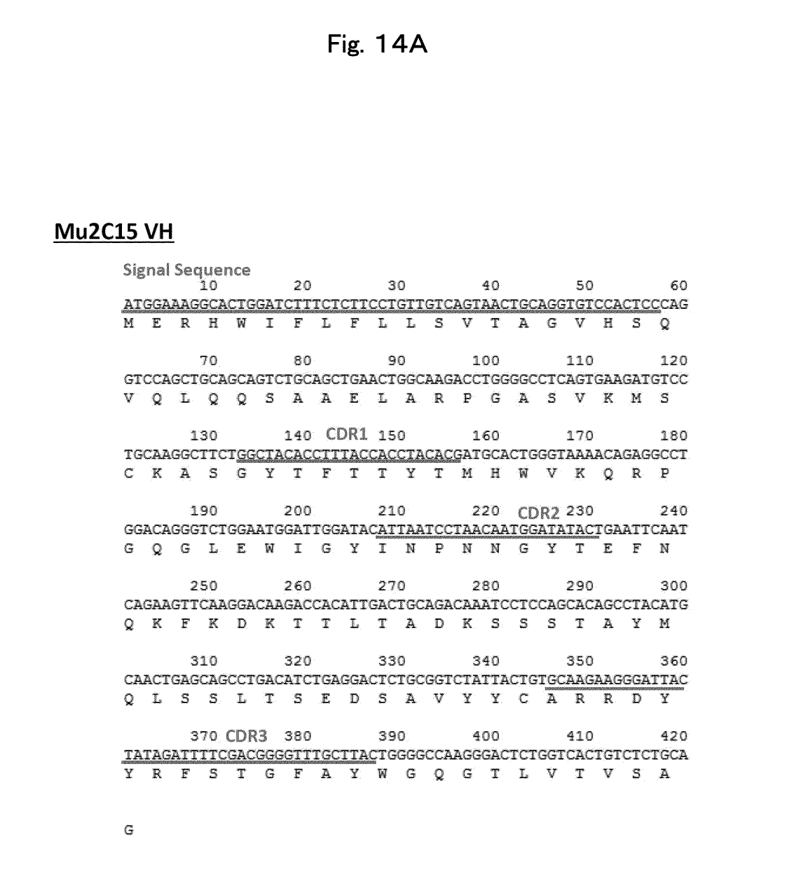

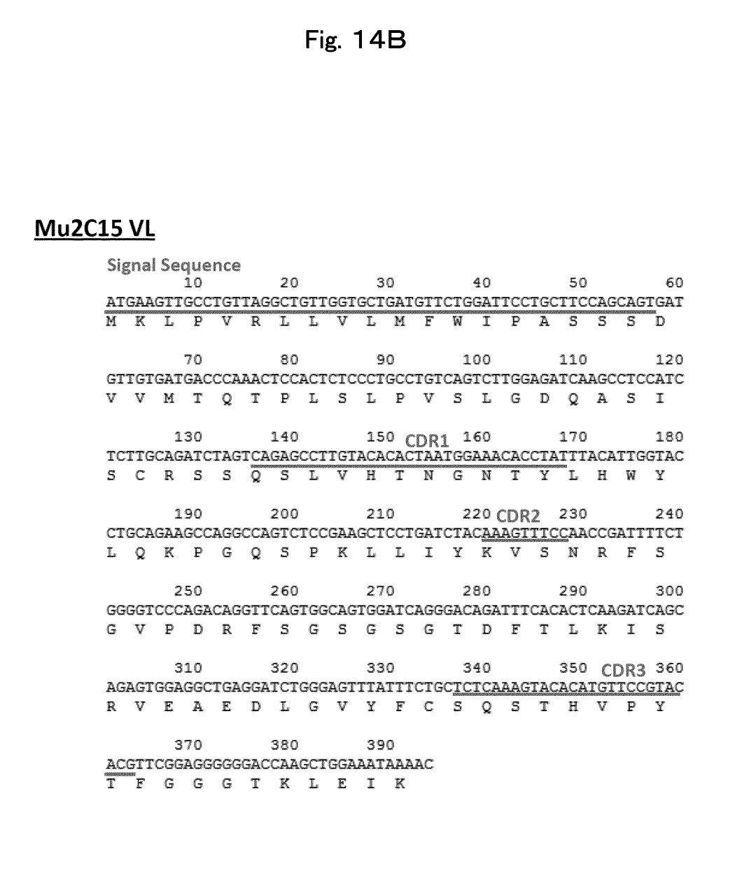

In addition, the nucleotide sequences encoding the above VH CDR1, VH CDR2 and VH CDR3 are show in SEQ ID NOs: 26, 28 and 30, respectively, while the nucleotide sequences encoding the above VL CDR1, VL CDR2 and VL CDR3 are shown in SEQ ID NOs: 32, 34 and 36, respectively. The positions of CDRs in VH and VL of the Mu2C15 antibody are shown in FIG. 14A and FIG. 14B.

### (5) Study on Binding Ability to Other LGR Family Proteins

### (5-1) Homology Analysis of LGR Family Proteins

As a representative example of the antibodies produced from the hybridoma cells obtained in Example 1 (7), the Mu2C15 antibody was used and analyzed for its binding ability to molecules belonging to the LGR family other than LGR6. Proteins having homology with the extracellular domain of human LGR6 are exemplified by human LGR4 and human LGR5. The amino acid sequence of human LGR4 is shown in SEQ ID NO: 39 and the amino acid sequence of human LGR5 is shown in SEQ ID NO: 41, each having homology with ECD2 described in Example 1 (3) and (4).

Amino acid sequence consisting of amino acids at positions 311 to 577 of human LGR4: (SEQ ID NO: 39)

Amino acid sequence consisting of amino acids at positions 319 to 563 of human LGR5: (SEQ ID NO: 41)

These amino acid sequences were analyzed for their homology with the amino acid sequence of ECD2 in human LGR6, indicating that the similarity was 78% between LGR6 and LGR4 and was 83% between LGR6 and LGR5, while the identity was 42% between LGR6 and LGR4 and was 51% between LGR6 and LGR5 (FIG. 1).

Amino acid sequence consisting of amino acids at positions 311 to 577 of human LGR4:

(SEQ ID NO: 39)
QFPNLTGTVHLESLTLTGTKISSIPNNLCQEQKMLRTLDLSYNNIRD
LPSFNGCHALEEISLQRNQIYQIKEGTFQGLISLRILDLSRNLIHEI
HSRAFATLGPITNLDVSFNELTSFPTEGLNGLNQLKLVGNFKLKEAL
AAKDFVNLRSLSVPYAYQCCAFWGCDSYANLNTEDNSLQDHSVAQEK
GTADAANVTSTLENEEHSQIIIHCTPSTGAFKPCEYLLGSWMIRLTV
WFIFLVALFFNLLVILTTFASCTSLPSSKLFI

Amino acid sequence consisting of amino acids at positions 319 to 563 of human LGR5:

(SEQ ID NO: 41)
TEFPDLTGTANLESLTLTGAQISSLPQTVCNQLPNLQVLDLSYNLLE
DLPSFSVCQKLQKIDLRHNEIYEIKVDTFQQLLSLRSLNLAWNKIAI
IHPNAFSTLPSLIKLDLSSNLLSSFPITGLHGLTHLKLTGNHALQSL
ISSENFPELKVIEMPYAYQCCAFGVCENAYKISNQWNKGDNSSMDDL
HKKDAGMFQAQDERDLEDFLLDFEEDLKALHSVQCSPSPGPFKPCEH
LLDGWLIRIG

### (5-2) Cloning of Genes for Partial Proteins of LGR Family Proteins

HT-29 cells were cultured and their RNA was extracted in the same manner as shown in Example 1 (2). After synthesis of cDNA, the following primers were used to amplify DNA comprising a nucleotide sequence (SEQ ID NO: 38) encoding amino acids at positions 311 to 577 of LGR4.

### Primer Sequences

LGR4-F:
(SEQ ID NO: 42)
CGTGAATTCGCAGCAGTTCCCCAATCTTAC
LGR4-R:
(SEQ ID NO: 43)
CGCAAAGCTTAGTAAGACGAATCATCCAGC

PCR reaction was conducted by preincubation at 95° C. for 10 minutes and subsequent 40 cycles of denaturation at 95° C. for 15 seconds and annealing/elongation at 58° C. for 1 minute to thereby amplify the desired gene fragment.

The resulting amplified fragment was cleaved with restriction enzymes (Hind III and EcoRI) at the restriction enzyme sites located on the primers and then integrated into pET32b vector (Invitrogen) to thereby obtain DNA comprising a nucleotide sequence encoding a partial protein of LGR4 having Trx-, S- and His-tags on the N-terminal side and a His-tag on the C-terminal side.

A nucleotide sequence (SEQ ID NO: 40) encoding amino acids at positions 319 to 563 of LGR5 was prepared by chemical synthesis (Medical & Biological Laboratories Co., Ltd., Japan), and then cleaved with restriction enzymes (BamHI and EcoRI) at the restriction enzyme sites designed outside of this nucleotide sequence and integrated into pET32a vector (Invitrogen) to thereby obtain DNA comprising a nucleotide sequence encoding a partial protein of LGR5 having Trx-, S- and His-tags on the N-terminal side and a His-tag on the C-terminal side.

### (5-3) Study on Binding Ability to LGR4 and LGR5 Partial Proteins

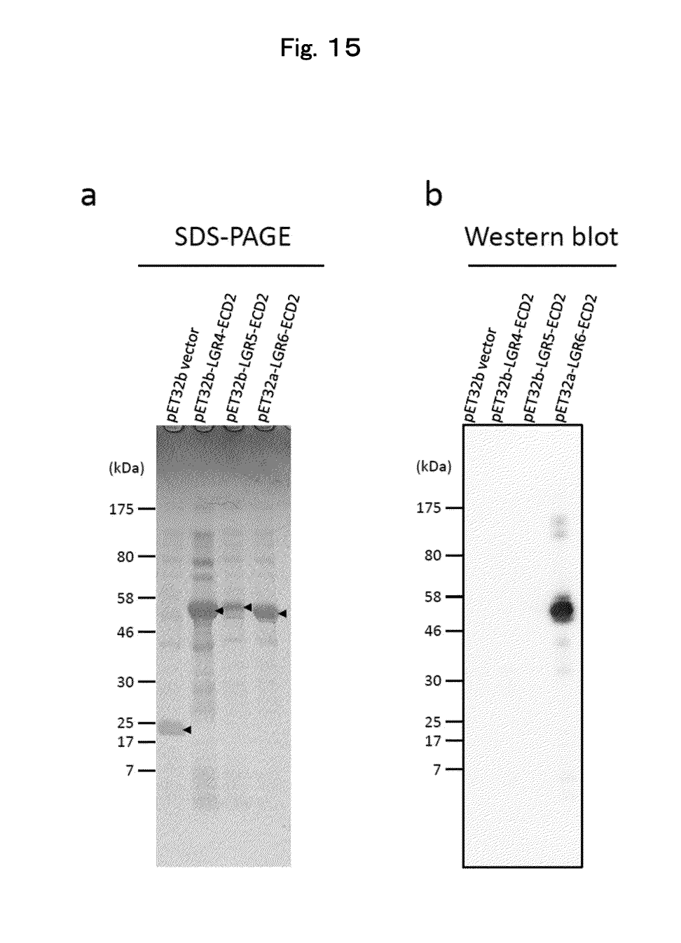

The vector comprising the nucleotide sequence encoding the partial protein of LGR6 described in Example 1. (2) and the vectors comprising the nucleotide sequences encoding the partial proteins of LGR4 and LGR5 prepared in (5-2) above were each used to transform BL21(DE3). Likewise, pET32b vector was also used to transform BL21(DE). After expression was induced in the same manner as shown in Example 1. (3), 5× Loading Buffer (10% SDS, 10 mM dithiotheritol, 20% glycorol, 200 mM Tris-HCl (pH 6.8), 0.05% bromophenol blue) was added in a volume of 5 μl relative to 20 μl of each cell culture solution, followed by heating at 95° C. for 5 minutes. Each sample was then provided for SDS-PAGE (FIG. 15a).

After electrophoresis, the proteins were transferred onto a PVDF membrane. Skimmed milk dissolved at a concentration of 5% (w/v) in TBS-T was used to block the membrane at room temperature for 30 minutes, and the membrane was washed twice with TBS-T. Among the monoclonal antibodies obtained in Example 1. (7), Mu2C15 was diluted to 1 μg/mL with TBS-T and reacted with the membrane for 1 hour at room temperature. After washing three times with TBS-T, anti-mouse IgG polyclonal antibody-HRP label was diluted 100,000-fold with TBS-T for use as a secondary antibody. This dilution was reacted with the membrane at room temperature for 30 minutes, followed by washing three times with TBS-T. The membrane was soaked in Immobilon and then wrapped, followed by signal detection with LAS-3000 (FIG. 15b)

These results indicated that the Mu2C15 antibody was reactive to the partial protein of LGR6, but was not reactive to the partial proteins of LGR4 and LGR5.

### Example 3

### Analysis of Clinical Tissue Samples

### (1) Immunohistological Staining Using Colorectal Cancer Tissues

The rate of LGR6 expression in colorectal cancer was analyzed by analysis of the reactivity between Mu2C15 and colorectal cancer tissue. In this example, human colorectal cancer tissue array slides (CDA3; SuperBiochips) were used for analysis.

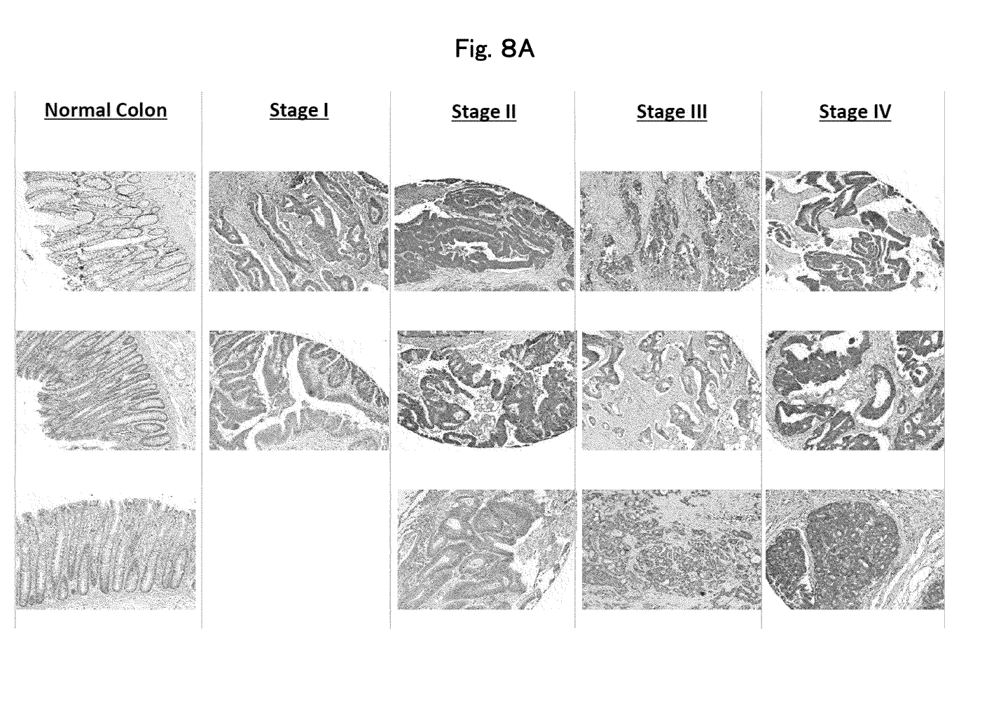

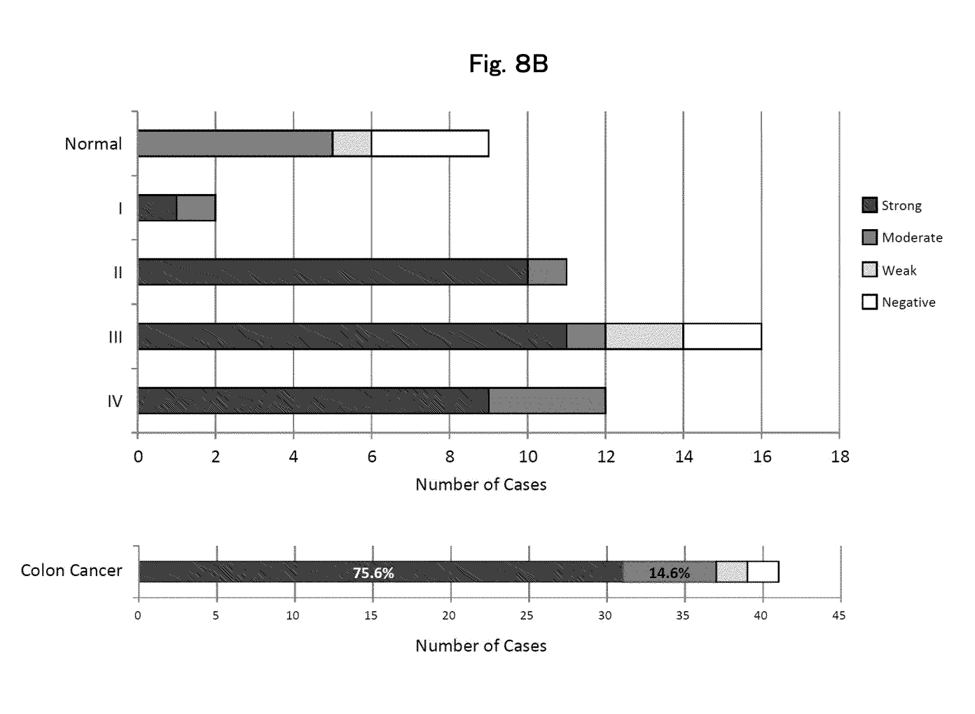

The colorectal cancer tissue array slides were deparaffinized by being soaked three times in xylene for 5 minutes. The slides were then soaked twice in 100% ethanol for 5 minutes and further soaked sequentially in 90% ethanol and 80% ethanol for 5 minutes each, and then soaked under running water for 2 minutes to hydrate the samples. The slides were soaked in 10 mM citrate buffer (pH 6.0) and microwaved at 600 W for 5 minutes. This operation was repeated three times in total to activate the antigen. The slides were treated with 3% hydrogen peroxide solution diluted in methanol for 10 minutes to thereby deactivate endogenous peroxidase activity. For the subsequent detection, a Histofine (M) kit (Nichirei Corporation, Japan) was used to conduct blocking, secondary antibody reaction, washing and detection reaction in accordance with the protocols attached to the kit. For primary antibody reaction, Mu2C15 was diluted to 5 or 10 μg/mL with TBS-T and reacted at room temperature for 1 hour. For color development, Impact DAB (Vector Laboratories) was used and reacted at room temperature for 5 minutes. The reacted slides were washed under running water and then counter stained with hematoxylin (Merck) and then sealed. The results of tissue staining are shown in FIG. 8A.

The degree of staining in immunohistological staining was ranked as strongly positive (Strong), positive (Moderate), weakly positive (Weak) or negative (Negative), and the results of immunohistological staining with Mu2C15 were statistically analyzed. The results obtained are shown in FIG. 8B.

In the case of normal colorectal mucosa, about half of the tissue samples used for measurement were almost not stained, and the remainder showed only weak staining. In contrast, in the case of colorectal cancer tissues, strongly positively stained tissues were already seen in stage I of cancer progression, and 90% or more of the samples in stage II used for measurement were found to show strongly positive results. This indicated that LGR6 was already expressed in early stages of colorectal cancer and 90% or more of the colorectal cancer tissue samples used for measurement showed positive or strongly positive results, and that LGR6 expression was able to be detected with high sensitivity when using the Mu2C15 antibody.

### (2) Immunohistological Staining Using Breast Cancer Tissues

The rate of LGR6 expression in breast cancer was analyzed by analysis of the reactivity between Mu2C15 and breast cancer tissue. For analysis, human breast cancer tissue array slides (FDA808b; USBiomax, BC081120; USBiomax, and CBA4; SuperBiochips) were used.

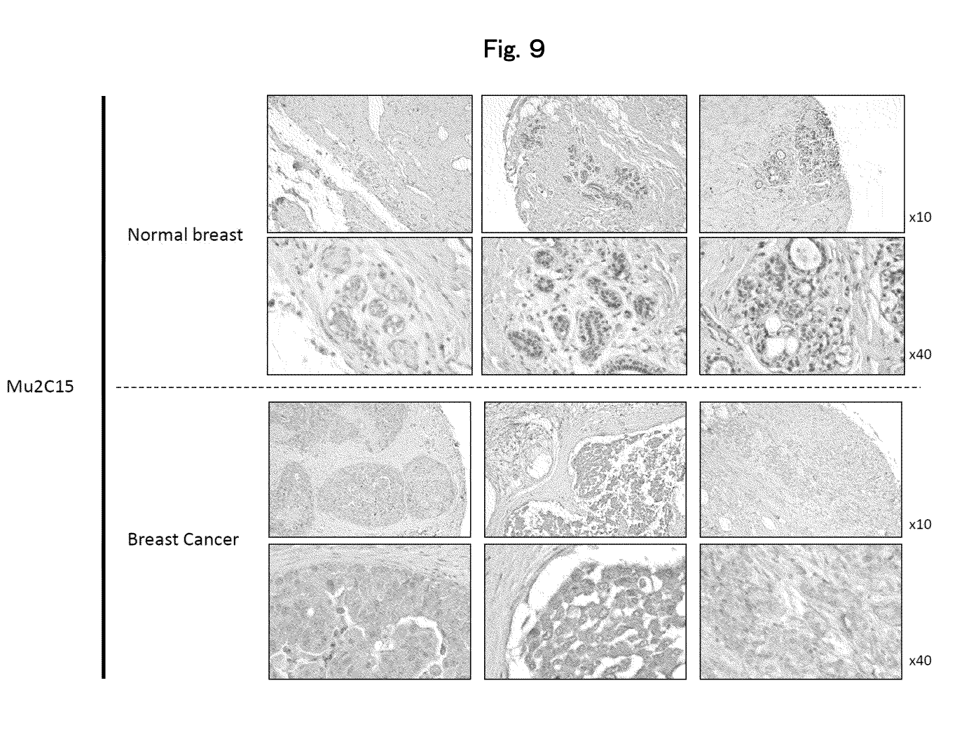

Immunostaining was conducted in the same manner as shown in Example 3. (1) above. The results of the experiment are shown in FIG. 9.

In the case of normal tissue, no staining was observed or glandular cells were positive in some samples. In contrast, in the case of breast cancer tissues, strongly positively stained tissues were already seen in stage I of cancer progression, and 80% or more of the samples used for measurement were found to show strongly positive or positive results. This indicated that LGR6 was already expressed in early stages of breast cancer and 90% or more of the breast cancer tissue samples used for measurement showed positive or strongly positive results, and that LGR6 expression was able to be detected with high sensitivity when using the Mu2C15 antibody.

These results indicated that LGR6 was a marker available for use in cancer diagnosis at least for both colorectal cancer and breast cancer. Moreover, it was also indicated that the antibody of the present invention was useful for detection and diagnosis of at least colorectal cancer and breast cancer.

(3) Comparison with HercepTest II

Her2 expression is used as an index to determine whether a patient should receive treatment with Herceptin, which is an existing molecular targeted drug. It is said that Her2-positive patients account for only 15% to 20% of all breast cancer patients. Her2 testing is conducted in the case of invasive breast cancer. For identification of Her2 expression, in vitro diagnostic agents are commercially available from several manufacturers. Among them, HercepTest II (DAKO) was used to analyze the positive rate of Her2 in the same tissue array slides as used in Example 3. (2) above, and a comparison was made with the positive rate of LGR6.

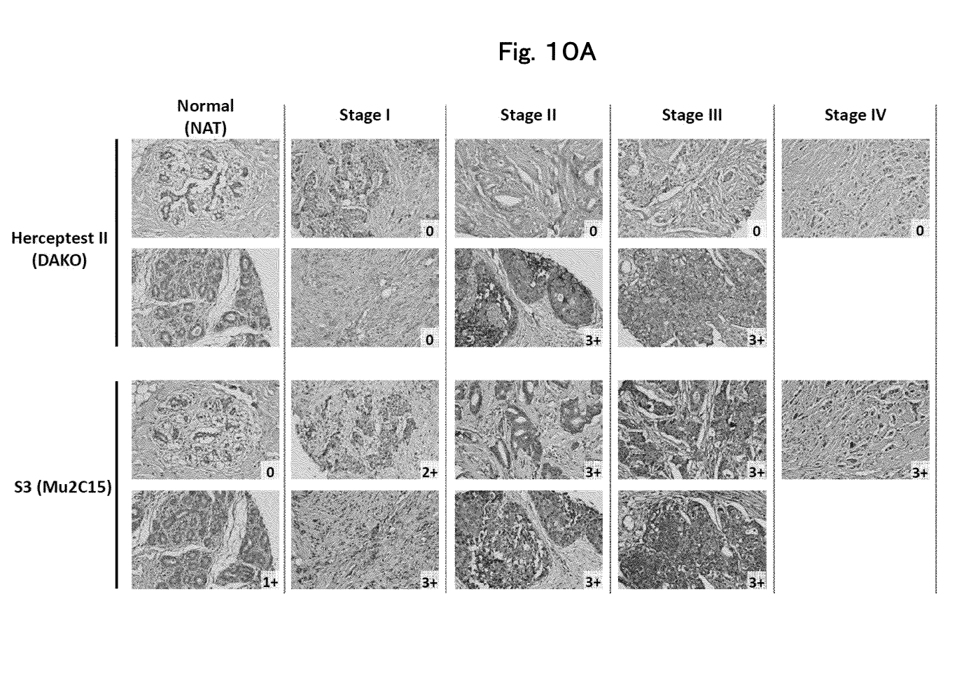

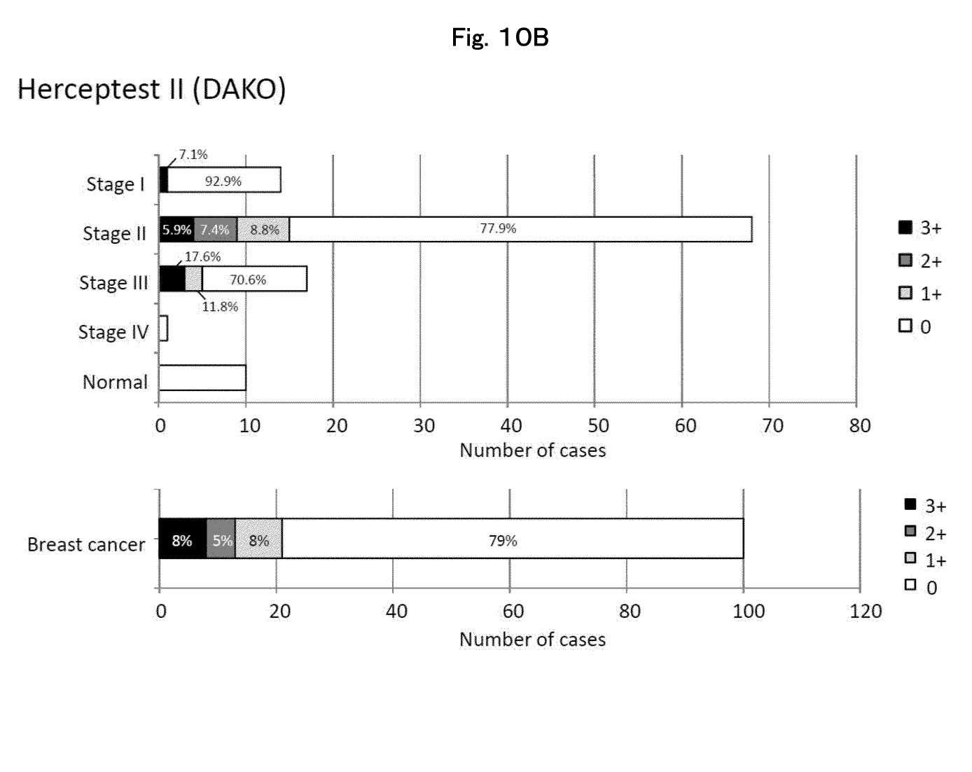

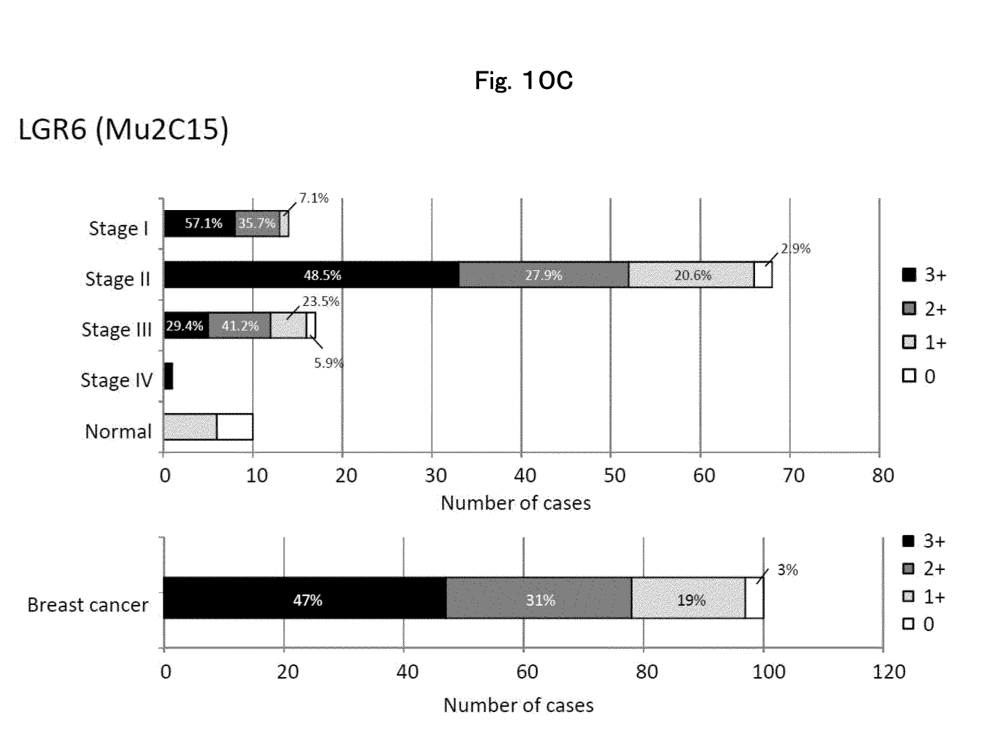

The results of immunostaining in breast cancer tissues at different stages are shown in FIG. 10A. Moreover, the results analyzed for the positive rate of Her2 and the positive rate of LGR6 are shown in FIG. 10B and FIG. 10C, respectively.

In the case of HercepTest II, among the samples used for measurement, those detected as being strongly positive (3+) and positive (2+) accounted for 13% (FIG. 10B, lower panel), whereas such samples accounted for 78% upon detection with the anti-LGR6 antibody (FIG. 10C, lower panel). Further, expression was analyzed for each stage. In stage I cases, only 7.1% were able to be detected by HercepTest II (FIG. 10B, upper panel), whereas 90% or more were able to be detected when using the anti-LGR6 antibody (FIG. 10C, upper panel).

These results indicated that breast cancer was able to be detected at stage I with high sensitivity when using the antibody of the present invention, and that the reagent of the present invention comprising this antibody was very effective for detection of breast cancer at early stages.

Moreover, it was also indicated that the grade of malignancy of breast cancer was able to be evaluated when using the antibody of the present invention.

As a monoclonal antibody binding to the extracellular domain of LGR6, the antibody under clone No. 2A3 (Abcam) is known. The titers of Mu2C15 and 2A3 were compared using colorectal cancer tissues.

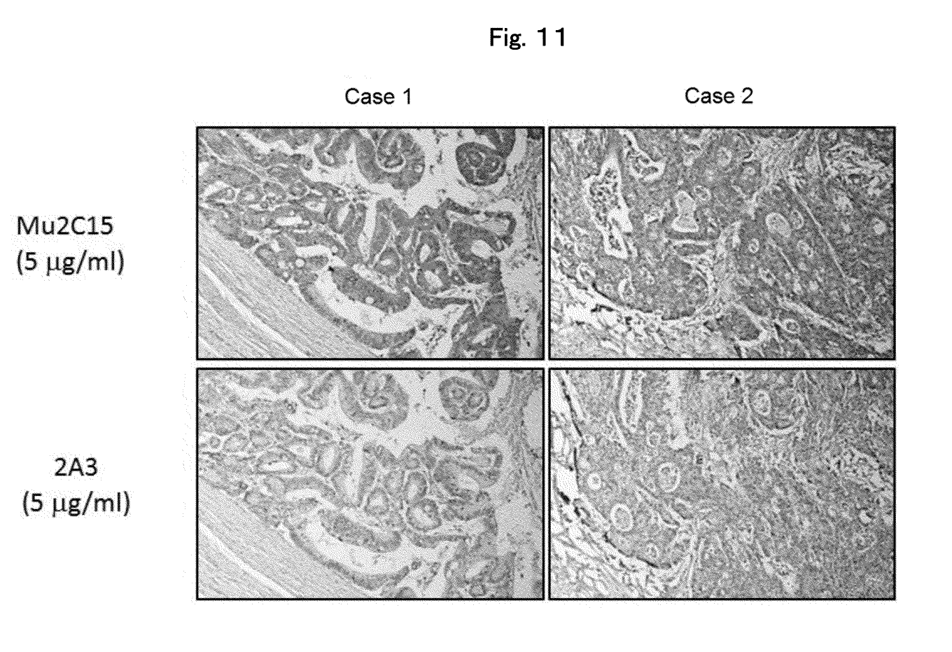

Immunostaining was conducted in the same manner as shown in Example 3. (1) above, such that the antibody concentration was adjusted to 5 μg/mL. The results of the experiment are shown in FIG. 11.

In colorectal cancer tissues from two cases, clear staining was observed when using Mu2C15, whereas no staining was observed when using 2A3.

This result indicated that the antibody of the present invention had a higher affinity and hence allowed cancer detection with higher sensitivity than the existing monoclonal antibody binding to the extracellular domain of LGR6.

### Example 4

### Construction of LGR6 Detection System

In some membrane proteins such as EGFR, it is known that their extracellular domains are released into blood (Molecular Cancer, Vol. 9, 166, 2010). Likewise, in the case of LGR6, it is also expected that cancer diagnosis is achieved by detection of an LGR6 extracellular domain segment in blood. Moreover, it is also assumed that the LGR6 protein is extracted and detected in a denatured state from feces. Thus, the monoclonal antibodies produced by the six lines of hybridoma cells obtained in Example 1. (7) were used to construct a method for detecting the extracellular domain of LGR6 with high sensitivity by sandwich ELISA.

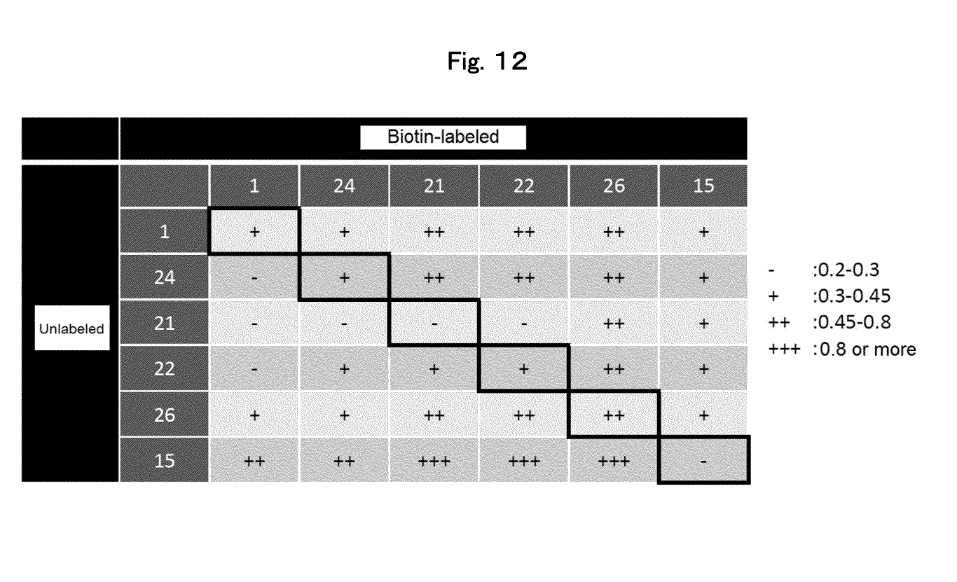

First, the monoclonal antibodies were labeled with biotin using a Biotin Labeling Kit-NH2 (Dojindo Laboratories, Japan) in accordance with the protocols attached thereto. Then, the monoclonal antibodies in an unlabeled state were dispensed into 96-well ELISA plates (Nunc) in an amount of 500 ng per well and immobilized on the plate surface by being allowed to stand overnight at 4° C. Then, after the plates were washed three times with 350 μL of PBS(−) containing 0.05% Tween 20 (hereinafter abbreviated as PBS-T), PBS-T containing 1% skimmed milk was dispensed in a volume of 300 μL per well, followed by blocking for 1 hour at room temperature. After washing with PBS-T, ECD2 was added at a concentration of 50 ng/mL and reacted for 1 hour at room temperature. After washing three times with 350 μL of PBS-T, the biotinylated antibodies prepared at a concentration of 0.5 mg/mL were each added and reacted for 1 hour at room temperature. After washing three times with 350 μL of PBS-T, a 5000-fold dilution of peroxidase-labeled streptavidin (Vector Labs) in TBS-T was added in 100 μL volumes and reacted for 1 hour at room temperature. The subsequent detection was conducted in the same manner as shown in Example 1. (6). The experimental results obtained are shown in FIG. 12.

The experimental results indicated that when Mu2C15 was immobilized and Mu2C21 or Mu2C22 was labeled with biotin for detection, ECD2 was able to be detected with low background and with high sensitivity.

Further, the combination of these antibodies was used to analyze the detection limit (critical value) for ECD2.

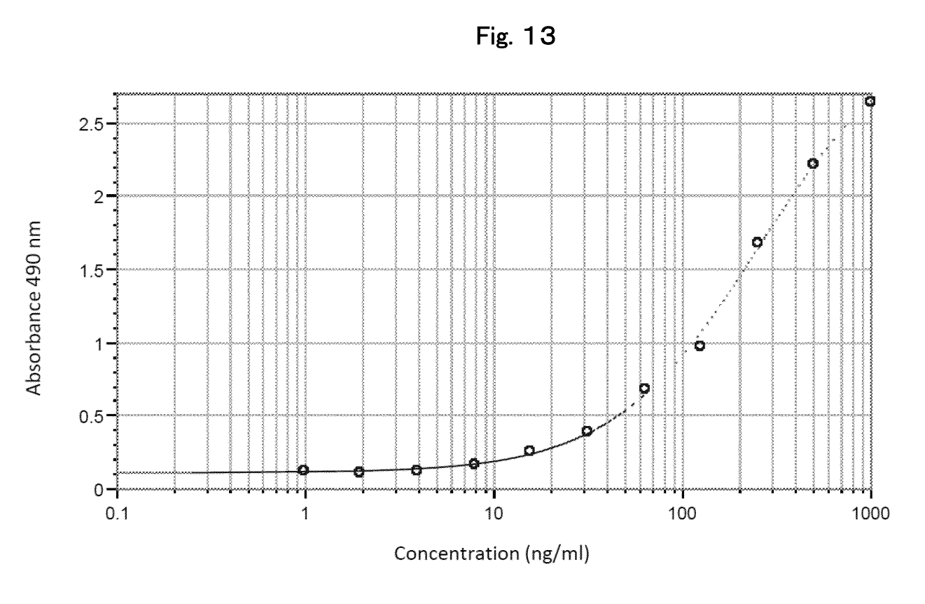

The experimental results obtained are shown in FIG. 13. As a result, it is indicated that a concentration greater than approximately 20 ng/mL would be sufficient for detection.

This result indicated that cancer was able to be detected with high accuracy and high sensitivity from biological samples (e.g., blood samples, feces) when using the antibody of the present invention.

### INDUSTRIAL APPLICABILITY

The present invention enables the detection of cancer with higher sensitivity than conventional antibodies.

### SEQUENCE LISTING FREE TEXT

SEQ ID NOs: 7 to 12: synthetic peptide

SEQ ID NOs: 13 to 21: synthetic DNA

SEQ ID NO: 22: synthetic DNA

SEQ ID NO: 23: synthetic peptide

SEQ ID NO: 24: synthetic DNA

SEQ ID NO: 25: synthetic peptide

SEQ ID NO: 26: synthetic DNA

SEQ ID NO: 27: synthetic peptide

SEQ ID NO: 28: synthetic DNA

SEQ ID NO: 29: synthetic peptide

SEQ ID NO: 30: synthetic DNA

SEQ ID NO: 31: synthetic peptide

SEQ ID NO: 32: synthetic DNA

SEQ ID NO: 33: synthetic peptide

SEQ ID NO: 34: synthetic DNA

SEQ ID NO: 35: synthetic peptide

SEQ ID NO: 36: synthetic DNA

SEQ ID NO: 37: synthetic peptide

SEQ ID NO: 38: synthetic DNA

SEQ ID NO: 39: synthetic peptide

SEQ ID NO: 40: synthetic DNA

SEQ ID NO: 41: synthetic peptide

SEQ ID NOs: 42 and 43: synthetic DNA

1. A reagent for detection or diagnosis of cancer, which comprises a monoclonal antibody against leucine-rich repeat containing G protein-coupled receptor 6 (LGR6) or a fragment of the antibody.

2. The reagent according to claim 1, wherein the antibody or fragment thereof binds to the extracellular region of leucine-rich repeat containing G protein-coupled receptor 6 (LGR6).

3. The reagent according to claim 2, wherein the extracellular region comprises the amino acid sequence shown in SEQ ID NO: 7, 8, 9 or 10.

4. The reagent according to claim 1, wherein the cancer is at least one selected from the group consisting of colorectal cancer, breast cancer, uterine cancer, gastric cancer, thyroid cancer, pancreatic cancer, brain tumor, cervical cancer, esophageal cancer, tongue cancer, lung cancer, small intestinal cancer, duodenal cancer, bladder cancer, kidney cancer, liver cancer, prostate cancer, uterine cervical cancer, ovarian cancer, gallbladder cancer, pharyngeal cancer, sarcoma, melanoma, leukemia, lymphoma and multiple myeloma.

5. The reagent according to claim 1, wherein the cancer is at least one selected from the group consisting of colorectal cancer, breast cancer, uterine cancer, gastric cancer, thyroid cancer, pancreatic cancer and leukemia.

6. The reagent according to claim 1, wherein the cancer is colorectal cancer or breast cancer.

7. A method for detection of cancer, which comprises the step of contacting a monoclonal antibody against leucine-rich repeat containing G protein-coupled receptor 6 (LGR6) or a fragment of the antibody with a biological sample taken from a subject to thereby detect LGR6 in the sample.

8. A kit for detection of cancer, which comprises a monoclonal antibody against leucine-rich repeat containing G protein-coupled receptor 6 (LGR6) or a fragment of the antibody.

9. A method for diagnosis of cancer, which comprises the step of contacting a monoclonal antibody against leucine-rich repeat containing G protein-coupled receptor 6 (LGR6) or a fragment of the antibody with a biological sample taken from a subject, and detecting LGR6 in the sample.

10. A monoclonal antibody against leucine-rich repeat containing G protein-coupled receptor 6 (LGR6) or a fragment of the antibody for use in the detection or diagnosis of cancer.


SEQUENCE LISTING (43 sequences)

SEQ ID NO: 1 - DNA, 3623 bp, Mus musculus
  CDS (137)..(3040)
aagtcgagcgggggcgttgcccaccgacggcgcagccctcgggcccgcccgggaccaggaggtgagccgcgcgcgcacaactccgtgcgctcgcccgtctgagcgcccgccaggtgccccgcagcccgccgccgagatgcacagcccgcctgggctcctggcgctgtggctttgcgctgtgctgtgcgcatcggcgcgcgcgggcagcgacccccagcctggcccggggcgtcccgcctgcccggctccctgccactgccaggaggacggcatcatgctgtccgctgactgctccgagctcgggctctcagtggtgcctgcggacctggaccccctgacggcttacctagacctcagtatgaacaacctcacggagcttcagccgggtctcttccaccacctgcgcttcctggaggagctgcggctctcagggaaccacctctcacacatcccgggacaggcattctctggcctccacagcctcaaaattctaatgctgcagagcaaccagctccgtgggatcccagcagaggcactatgggagctgcccagcctgcagtcgctgcgcctagatgctaatctcatctccctggtccctgagagaagctttgaggggctctcctccctccgccacctctggctggatgacaatgcactcaccgagatccccgtcagagctctcaacaaccttcctgccctacaagccatgaccttggctctcaaccatatccgccacatccctgactatgccttccagaacctcaccagtcttgtggtgctgcatctacataacaaccgcatccagcatgtggggacccacagcttcgaggggctgcacaatctggagacactagacctgaactataatgagctgcaggagttccccttggctatccggaccctgggcaggctgcaggaattgggtttccataacaacaacatcaaggctatcccagagaaagccttcatgggcagccctctcctgcagacaatacatttttatgacaacccaatccagtttgtgggaaggtcagcattccagtacctgtctaaactgcatacgctatctttgaatggtgccactgatatccaagagttcccagacctcaaaggcaccactagcctggagatcctgaccctgacccgtgcgggcatcagactgctcccaccgggagtgtgccaacagctgcctaggctccgaatcctggagctgtctcataatcagatcgaggagttacccagcctgcacaggtgtcagaagctggaggaaattggcctccgacataaccggatcaaggaaattggtgcagataccttcagccagctgggctccttgcaagctttagacctgagttggaatgccatccgtgccatccaccctgaggctttctcaacccttcgatccttggttaagctggacctgactgacaaccagctgaccacactgcccctggctgggctgggaggcctgatgcacctgaagctcaaagggaacttggccctgtctcaggccttctccaaggacagtttcccaaaactgaggatcctggaggtgccctacgcctaccagtgctgtgcctacggcatctgtgccagcttcttcaagacctctgggcagtggcaggccgaggactttcatccagaggaagaggaggcaccaaagaggcccctgggtctccttgctggacaagctgagaaccactatgacctagacctggatgagctccagatggggacagaggactcaaagccacaccccagtgtccagtgcagccctgttccaggccccttcaagccctgcgagcacctctttgagagctggggcatccgccttgctgtgtgggccatcgtgttactctccgtactctgtaacgggctggtgctgctgacagtctttgccagcggacccagcccgctgtcccccgtcaagcttgtggtgggtgcgatggcaggcgccaacgccctgtcgggcatttcctgtggtctcctggcctcggtggacgccttgacctatggtcagttcgctgagtatggagcccgctgggagagcggtctgggctgccaggctacgggcttcctggctgtcctgggttcagaggcgtcggtgctgctgctcacactggcggccgtgcagtgcagcatctccgtgacctgcgtccgagcctacgggaaggcgccgtcgcctggcagcgtccgcgcaggcgcactgggatgcctggcgctggccgggctggccgcagcactgccgctggcctcggtgggagagtatggcgcctccccactctgcctgccctacgccccacccgagggccggccggctgccctgggcttcgctgtagccctggtgatgatgaactcgctctgcttcctggtggtggccggcgcctacatcaagctctactgtgacctgccacggggtgactttgaggccgtgtgggactgcgccatggtgcgccacgtggcctggctcatctttgcagatggcctcctctactgccccgtggccttcctcagctttgcctctatgctgggcctcttccctgtcacccccgaggctgtcaagtcagtccttctggtggtgctgcctctgcctgcctgcctcaacccactgctctacctgctcttcaaccctcacttccgggatgaccttcggcggctctggccaagccctcggtccccagggcccctagcctacgctgcagccggtgagctggagaagagctcctgcgactccacccaagcgctggtggctttctcagatgtggatcttattctggaagcttctgaggctgggcagcctcctgggctagagacctatggcttcccttcagtgaccctcatctcccgacatcagccgggggctaccaggctggagggaaaccattttgtagagtctgatggaaccaagtttgggaacccacaacctcccatgaagggagaactgctgctgaaggcagagggagccactttggcaggctgtggctcttccgtgggtggagccctctggccctctggctctctctttgcctctcacttgtaaatatccctctctgtttgtcctcttcccgtccaatgatggctgcttataaaagaaagacaactccaactccatagcaagatggccaacacctctgactccattgttctctctccacgacccctaaccaatgagtgcttccaagtcttgctttgtcttggccttcagcttcactttcaccctgggccttctctgtccaatccaatacttctgacagaggcctgggaaatttgcataggagaaaggagaaaagcaaaagacagtgaaggttatggggccctgacagagccatgatcagtaagtgcagagtgatggggaggtctcacagagcatgacactggaagacaactaccaaagacattggagagtctcccctgtgacatatagaatataaaatgtgttttgtgttccattaatcttgacctatgctgtgccaaagtgcttcctgttaaaatacactttggaagacattgcatattatgtccctttcttagagtcatggggtcatgttagtgtctaaaaggtcctgagaattaggactttttggcttaattactacagcaattaaacttaattatttgatgat

SEQ ID NO: 2 - protein, 967 aa, Mus musculus
MHSPPGLLALWLCAVLCASARAGSDPQPGPGRPACPAPCHCQEDGIMLSADCSELGLSVVPADLDPLTAYLDLSMNNLTELQPGLFHHLRFLEELRLSGNHLSHIPGQAFSGLHSLKILMLQSNQLRGIPAEALWELPSLQSLRLDANLISLVPERSFEGLSSLRHLWLDDNALTEIPVRALNNLPALQAMTLALNHIRHIPDYAFQNLTSLVVLHLHNNRIQHVGTHSFEGLHNLETLDLNYNELQEFPLAIRTLGRLQELGFHNNNIKAIPEKAFMGSPLLQTIHFYDNPIQFVGRSAFQYLSKLHTLSLNGATDIQEFPDLKGTTSLEILTLTRAGIRLLPPGVCQQLPRLRILELSHNQIEELPSLHRCQKLEEIGLRHNRIKEIGADTFSQLGSLQALDLSWNAIRAIHPEAFSTLRSLVKLDLTDNQLTTLPLAGLGGLMHLKLKGNLALSQAFSKDSFPKLRILEVPYAYQCCAYGICASFFKTSGQWQAEDFHPEEEEAPKRPLGLLAGQAENHYDLDLDELQMGTEDSKPHPSVQCSPVPGPFKPCEHLFESWGIRLAVWAIVLLSVLCNGLVLLTVFASGPSPLSPVKLVVGAMAGANALSGISCGLLASVDALTYGQFAEYGARWESGLGCQATGFLAVLGSEASVLLLTLAAVQCSISVTCVRAYGKAPSPGSVRAGALGCLALAGLAAALPLASVGEYGASPLCLPYAPPEGRPAALGFAVALVMMNSLCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLWPSPRSPGPLAYAAAGELEKSSCDSTQALVAFSDVDLILEASEAGQPPGLETYGFPSVTLISRHQPGATRLEGNHFVESDGTKFGNPQPPMKGELLLKAEGATLAGCGSSVGGALWPSGSLFASHL

SEQ ID NO: 3 - DNA, 3673 bp, Rattus norvegicus
  CDS (131)..(3028)
agcgggggcggtccccaccgacggcgcagccctcgggcccgcccggaaccaggaggtgagccgcgcgctcacagctccgtgcgcccgcccgtcccagcgcccgccaggtgccccaaagcctgccgccgagatgcacagctcgccgttgctcctggcgctgtgtctttgcgttgtgctgtgcgcattggcgcgcgcgggcagcgacccccagcctggcccggggcgtcccgcctgccccgctccctgccactgccaggaggatggcatcatgctgtcagctgactgctccgagctcggtctctcagaggtgccagcggacctggaccccctgacggcttacctagacctcagtatgaacaacctcacggagcttcagccgggtctcttccaccacctgcgcttcctggaggagctgcggctctcggggaaccacctctcacacatcccaagacaggcattctctggcctccacagcctcaaaattctgatgctgcagagcaaccagctccgagggatcccagcagaggcgctgtgggagctgcccagcctgcagtcgctgcgcctagatgctaacctcatctccctggtccctgagagaagctttgaggggctgtcctccctccgccacctctggttggatgacaatgcactcaccgagatccctgtcagagctctcaacaaccttcctgccctacaggccatgaccttggctctcaaccgcatccgccacatccctgactatgccttccagaacctcaccagtcttgtggtgctgcatctacataacaaccgcatccagcatgtggggacccacagcttcgaggggctgcacaatctggagacactagacttgaactataatgagctgcaggagttccccgtggctatccggaccctgggcaggctgcaggaattgggtttccacaacaacaacatcaaggctatcccagagaaggccttcatgggcaaccctctcctgcagacaatacacttttatgacaacccgatccagtttgtgggaaggtcagcattccagtacctgtctaaactgcatacgctatccttgaatggtgccactgacatccaggagttcccagacctcaaaggcaccactagcctggagattctgaccctgacccgtgcaggcatcagactgctcccagcgggaatgtgtcaacagctgcctaggctccgaatcctggagctgtctcataatcagattgaggagctacccagcctgcacaggtgtcagaagttggaggaaattggcctccagcataaccggatctgggaaattggtgcagataccttcagccagctgagttccttgcaagctctagatctgagttggaatgccatccgtgccattcaccccgaggctttctcaacccttcgatccttggttaagctggacctgactgacaaccagctgaccacactgcccctggctgggctgggaggcctgatgcacctgaagctcaaggggaacttggccctgtctcaggccttctccaaggacagtttccccaaactgaggatcctggaggtgccttacgcctaccagtgctgtgcctatggcctctgtgccaacttcttcaagacctctgggcagtggcaggccgaggactttcatgcagaggaagaagaggcaccaaagaggcccctgggtctccttgctggacaagctgagaaccactatgacctagacctggatgagctccagatggagacagaggactcaaagccacaccccagtgtccagtgcagccctgttccaggccccttcaagccctgcgagcacctctttgagagctggggcatccgccttgctgtgtgggccatcgtgctgctctcggtactctgtaacgggctggtgctgcttacagtctttgctggcgggcccagcccgttgtctccggtcaagcttgtggtgggtgcgatcgctggcgccaacgccctaacgggcatttcctgcggcctcctggcctcagtggacgccttgaccttcggccagttcgcggagtatggagcccgctgggagaccgggctaggctgccaggctacgggcttcctagccgtcctggggtcggaggcgtcggtgctgctgctcacgctggcggccgtgcagtgcagcgtctctgtgacctgcgtccgggcctacgggaaggcgccgtcgcccggtagcgtccgcgcaggcgcactgggatgcttggcgctggctgggctggccgccgcgctgccgctggcctcggtgggggagtatggtgcctccccgctctgcctgccctacgcccctcccgagggccgcccggccgccctaggcttcgccgtagccctggtgatgatgaactcgctctgcttcctcgtggtggccggtgcctacatcaagctctactgcgacctgcctcggggtgactttgaggccgtgtgggactgcgccatggtgcggcatgtggcctggctcatctttgcagatggccttctctactgccccgtggccttcctcagctttgcctccatgctgggcctcttcccggtcacccctgaggctgtcaagtcggttcttctggtggtgttgcctctgcccgcctgcctgaacccactgctctacctgctcttcaaccctcacttccgggaagaccttcggaggctctggccgagccctcggtccccagggcctctagcttatactgcagccggtgagctggagaagagctcctgcgactccacccaagccctggtggctttctctgatgtggatcttattctggaagcttctgaggctgggcagcctcctgggctagagacctatggcttcccttcagtgaccctcatctcccgtcatcagccaggggctaccaggctggagggaaaccattttgtggagcctgatggaaccaagttagggaacccacaacctcccatgaatgaagaactgctgctgagggcagagggagccacactggcagactgttctgcaggtggagccctctggccctctggccctctctttgcctctcacttgtaaataacccccccccccaatttgtcctccctcctcccaatgatggctgcttataaaagaaagacaactacagctccgcagcagaatggccaacgcccctggctccattgttcaccgtggccataacaaggtgtgcttccaggtcttgctttgtcttggccttcagcttcacggtcaccctgggccttccctgtccaatccgagtatgtatgacagagacctgggaaatttacttaggagaaaggaggaaagcaaaagacagtgaaggctgtgggaggctgacagagttgcgaccagtgagttacacagtgagcggggaggcctcacagagaaaggcactggaaggcaactaccaaagacattggagtgtcccgcctacggcgtgtggatagcatagaaaacgtgttctgcgtcccaccaatcttgacctatgctgtgccaaagtgcttccagttaagatacgctttggcagacattgcatattatgcccctttcttagagtcatggggtcatgttagtgtctaaaaggtcctaagaattaggaccttttggcttaatgatgacagcagttaaacgtacttatttgatgataggtttctgtttgcttttcatggaacgcttacaaatctctcgcagaacttagcacgg

SEQ ID NO: 4 - protein, 965 aa, Rattus norvegicus
MHSSPLLLALCLCVVLCALARAGSDPQPGPGRPACPAPCHCQEDGIMLSADCSELGLSEVPADLDPLTAYLDLSMNNLTELQPGLFHHLRFLEELRLSGNHLSHIPRQAFSGLHSLKILMLQSNQLRGIPAEALWELPSLQSLRLDANLISLVPERSFEGLSSLRHLWLDDNALTEIPVRALNNLPALQAMTLALNRIRHIPDYAFQNLTSLVVLHLHNNRIQHVGTHSFEGLHNLETLDLNYNELQEFPVAIRTLGRLQELGFHNNNIKAIPEKAFMGNPLLQTIHFYDNPIQFVGRSAFQYLSKLHTLSLNGATDIQEFPDLKGTTSLEILTLTRAGIRLLPAGMCQQLPRLRILELSHNQIEELPSLHRCQKLEEIGLQHNRIWEIGADTFSQLSSLQALDLSWNAIRAIHPEAFSTLRSLVKLDLTDNQLTTLPLAGLGGLMHLKLKGNLALSQAFSKDSFPKLRILEVPYAYQCCAYGLCANFFKTSGQWQAEDFHAEEEEAPKRPLGLLAGQAENHYDLDLDELQMETEDSKPHPSVQCSPVPGPFKPCEHLFESWGIRLAVWAIVLLSVLCNGLVLLTVFAGGPSPLSPVKLVVGAIAGANALTGISCGLLASVDALTFGQFAEYGARWETGLGCQATGFLAVLGSEASVLLLTLAAVQCSVSVTCVRAYGKAPSPGSVRAGALGCLALAGLAAALPLASVGEYGASPLCLPYAPPEGRPAALGFAVALVMMNSLCFLVVAGAYIKLYCDLPRGDFEAVWDCAMVRHVAWLIFADGLLYCPVAFLSFASMLGLFPVTPEAVKSVLLVVLPLPACLNPLLYLLFNPHFREDLRRLWPSPRSPGPLAYTAAGELEKSSCDSTQALVAFSDVDLILEASEAGQPPGLETYGFPSVTLISRHQPGATRLEGNHFVEPDGTKLGNPQPPMNEELLLRAEGATLADCSAGGALWPSGPLFASHL

SEQ ID NO: 5 - DNA, 3458 bp, Homo sapiens
  CDS (1)..(2904)
atgcccagcccgccggggctccgggcgctatggctttgcgccgcgctgtgcgcttcccggagggccggcggcgccccccagcccggcccggggcccaccgcctgcccggccccctgccactgccaggaggacggcatcatgctgtctgccgactgctctgagctcgggctgtccgccgttccgggggacctggaccccctgacggcttacctggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccccgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgtccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcgctgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagctttgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtccctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttggaagagattacacatgatgtctttttcttagagattcacagtgcatgttagtgtaataaagagataagtcctacagta

SEQ ID NO: 6 - protein, 967 aa, Homo sapiens
MPSPPGLRALWLCAALCASRRAGGAPQPGPGPTACPAPCHCQEDGIMLSADCSELGLSAVPGDLDPLTAYLDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPVPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 7 - protein, 543 aa, Artificial
  synthetic peptide
APQPGPGPTACPAPCHCQEDGIMLSADCSELGLSAVPGDLDPLTAYLDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLA

SEQ ID NO: 8 - protein, 477 aa, Artificial
  synthetic peptide
FLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLA

SEQ ID NO: 9 - protein, 249 aa, Artificial
  synthetic peptide
QEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLA

SEQ ID NO: 10 - protein, 124 aa, Artificial
  synthetic peptide
GLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLA

SEQ ID NO: 11 - protein, 828 aa, Artificial
  synthetic peptide
MRLEGEGRSARAGQNLSRAGSARRGAPRDLSMNNLTELQPGLFHHLRFLEELRLSGNHLSHIPGQAFSGLYSLKILMLQNNQLGGIPAEALWELPSLQSLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPVPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 12 - protein, 915 aa, Artificial
  synthetic peptide
MGRPRLTLVCQVSIIISARDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPVPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 13 - DNA, 747 bp, Artificial
  synthetic DNA
caggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccccgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggcc

SEQ ID NO: 14 - DNA, 37 bp, Artificial
  synthetic DNA
tttgaattcgatgcaggagtttccagatctcaaaggc

SEQ ID NO: 15 - DNA, 33 bp, Artificial
  synthetic DNA
tttgcggccgccacgtgtgaagcaaaggccaag

SEQ ID NO: 16 - DNA, 45 bp, Artificial
  synthetic DNA
ctaatacgactcactatagggcaagcagtggtatcaacgcagagt

SEQ ID NO: 17 - DNA, 22 bp, Artificial
  synthetic DNA
ctaatacgactcactatagggc

SEQ ID NO: 18 - DNA, 34 bp, Artificial
  synthetic DNA
ccaggggccagtggatagacagatgggggtgtcg

SEQ ID NO: 19 - DNA, 26 bp, Artificial
  synthetic DNA
gcacctccagatgttaactgctcact

SEQ ID NO: 20 - DNA, 20 bp, Artificial
  synthetic DNA
gtcacgacgttgtaaaacga

SEQ ID NO: 21 - DNA, 20 bp, Artificial
  synthetic DNA
atttaggtgacactatagaa

SEQ ID NO: 22 - DNA, 421 bp, Artificial
  synthetic DNA
atggaaaggcactggatctttctcttcctgttgtcagtaactgcaggtgtccactcccaggtccagctgcagcagtctgcagctgaactggcaagacctggggcctcagtgaagatgtcctgcaaggcttctggctacacctttaccacctacacgatgcactgggtaaaacagaggcctggacagggtctggaatggattggatacattaatcctaacaatggatatactgaattcaatcagaagttcaaggacaagaccacattgactgcagacaaatcctccagcacagcctacatgcaactgagcagcctgacatctgaggactctgcggtctattactgtgcaagaagggattactatagattttcgacggggtttgcttactggggccaagggactctggtcactgtctctgcag

SEQ ID NO: 23 - protein, 140 aa, Artificial
  synthetic peptide
MERHWIFLFLLSVTAGVHSQVQLQQSAAELARPGASVKMSCKASGYTFTTYTMHWVKQRPGQGLEWIGYINPNNGYTEFNQKFKDKTTLTADKSSSTAYMQLSSLTSEDSAVYYCARRDYYRFSTGFAYWGQGTLVTVSA

SEQ ID NO: 24 - DNA, 394 bp, Artificial
  synthetic DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacactaatggaaacacctatttacattggtacctgcagaagccaggccagtctccgaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaac

SEQ ID NO: 25 - protein, 131 aa, Artificial
  synthetic peptide
MKLPVRLLVLMFWIPASSSDVVMTQTPLSLPVSLGDQASISCRSSQSLVHTNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 26 - DNA, 24 bp, Artificial
  synthetic DNA
ggctacacctttaccacctacacg

SEQ ID NO: 27 - protein, 8 aa, Artificial
  synthetic peptide
GYTFTTYT

SEQ ID NO: 28 - DNA, 24 bp, Artificial
  synthetic DNA
attaatcctaacaatggatatact

SEQ ID NO: 29 - protein, 8 aa, Artificial
  synthetic peptide
INPNNGYT

SEQ ID NO: 30 - DNA, 42 bp, Artificial
  synthetic DNA
gcaagaagggattactatagattttcgacggggtttgcttac

SEQ ID NO: 31 - protein, 14 aa, Artificial
  synthetic peptide
ARRDYYRFSTGFAY

SEQ ID NO: 32 - DNA, 33 bp, Artificial
  synthetic DNA
cagagccttgtacacactaatggaaacacctat

SEQ ID NO: 33 - protein, 11 aa, Artificial
  synthetic peptide
QSLVHTNGNTY

SEQ ID NO: 34 - DNA, 9 bp, Artificial
  synthetic DNA
aaagtttcc

SEQ ID NO: 35 - protein, 3 aa, Artificial
  synthetic peptide
KVS

SEQ ID NO: 36 - DNA, 27 bp, Artificial
  synthetic DNA
tctcaaagtacacatgttccgtacacg

SEQ ID NO: 37 - protein, 9 aa, Artificial
  synthetic peptide
SQSTHVPYT

SEQ ID NO: 38 - DNA, 801 bp, Artificial
  synthetic DNA
cagttccccaatcttacaggaactgtccacctggaaagtctgactttgacaggtacaaagataagcagcatacctaataatttgtgtcaagaacaaaagatgcttaggactttggacttgtcttacaataatataagagaccttccaagttttaatggttgccatgctctggaagaaatttctttacagcgtaatcaaatctaccaaataaaggaaggcacctttcaaggcctgatatctctaaggattctagatctgagtagaaacctgatacatgaaattcacagtagagcttttgccacacttgggccaataactaacctagatgtaagtttcaatgaattaacttcctttcctacggaaggcctgaatgggctaaatcaactgaaacttgtgggcaacttcaagctgaaagaagccttagcagcaaaagactttgttaacctcaggtctttatcagtaccatatgcttatcagtgctgtgcattttggggttgtgactcttatgcaaatttaaacacagaagataacagcctccaggaccacagtgtggcacaggagaaaggtactgctgatgcagcaaatgtcacaagcactcttgaaaatgaagaacatagtcaaataattatccattgtacaccttcaacaggtgcttttaagccctgtgaatatttactgggaagctggatgattcgtcttactgtgtggttcattttcttggttgcattatttttcaacctgcttgttattttaacaacatttgcatcttgtacatcactgccttcgtccaaattgtttata

SEQ ID NO: 39 - protein, 267 aa, Artificial
  synthetic peptide
QFPNLTGTVHLESLTLTGTKISSIPNNLCQEQKMLRTLDLSYNNIRDLPSFNGCHALEEISLQRNQIYQIKEGTFQGLISLRILDLSRNLIHEIHSRAFATLGPITNLDVSFNELTSFPTEGLNGLNQLKLVGNFKLKEALAAKDFVNLRSLSVPYAYQCCAFWGCDSYANLNTEDNSLQDHSVAQEKGTADAANVTSTLENEEHSQIIIHCTPSTGAFKPCEYLLGSWMIRLTVWFIFLVALFFNLLVILTTFASCTSLPSSKLFI

SEQ ID NO: 40 - DNA, 735 bp, Artificial
  synthetic DNA
actgaatttcctgatttaactggaactgcaaacctggagagtctgactttaactggagcacagatctcatctcttcctcaaaccgtctgcaatcagttacctaatctccaagtgctagatctgtcttacaacctattagaagatttacccagtttttcagtctgccaaaagcttcagaaaattgacctaagacataatgaaatctacgaaattaaagttgacactttccagcagttgcttagcctccgatcgctgaatttggcttggaacaaaattgctattattcaccccaatgcattttccactttgccatccctaataaagctggacctatcgtccaacctcctgtcgtcttttcctataactgggttacatggtttaactcacttaaaattaacaggaaatcatgccttacagagcttgatatcatctgaaaactttccagaactcaaggttatagaaatgccttatgcttaccagtgctgtgcatttggagtgtgtgagaatgcctataagatttctaatcaatggaataaaggtgacaacagcagtatggacgaccttcataagaaagatgctggaatgtttcaggctcaagatgaacgtgaccttgaagatttcctgcttgactttgaggaagacctgaaagcccttcattcagtgcagtgttcaccttccccaggccccttcaaaccctgtgaacacctgcttgatggctggctgatcagaattgga

SEQ ID NO: 41 - protein, 245 aa, Artificial
  synthetic peptide
TEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIG

SEQ ID NO: 42 - DNA, 30 bp, Artificial
  synthetic DNA
cgtgaattcgcagcagttccccaatcttac

SEQ ID NO: 43 - DNA, 30 bp, Artificial
  synthetic DNA
cgcaaagcttagtaagacgaatcatccagc